SEQUENCE LISTING (6 sequences)

SEQ ID NO: 1 - DNA, 1858 bp, Aspergillus fumigatus
atgttttcgtcgctcttgaaccgtggagctttgctcgcggttgtttctctcttgtcctcttccgttgctgccgaggtttttgagaagctgtccgcggtgccacagggtttgttctcccgaccccccgcctcttacgtcgtgactgacgagaacaggatggaaatactcccacacccctagtgaccgcgatcccattcgcctccagattgccctgaagcaacatgatgtcgaaggttttgagaccgccctcctggaaatgtccgatccctaccacccaaactatggcaagcactttcaaactcacgaggagatgaagcggatgctgctgcccacccaggaggcggtcgagtccgtccgcggctggctggagtccgctggaatctcggatatcgaggaggatgcagactggatcaagttccgcacaaccgttggcgtggccaatgacctgctggacgccgacttcaagtggtacgtgaacgaggtgggccacgttgagcgcctgaggaccctggcatactcgctcccgcagtcggtcgcgtcgcacgtcaacatggtccagcccaccacgcggttcggacagatcaagcccaaccgggcgaccatgcgcggtcggcccgtgcaggtggatgcggacatcctgtccgcggccgttcaagccggcgacacctccacttgcgatcaggtcatcacccctcagtgcctcaaggatctgtacaatatcggcgactacaaggccgaccccaacgggggcagcaaggtcgcgtttgccagtttcctggaggaatacgcccgctacgacgatctggccaagttcgaggagaagctggccccgtacgccattggacagaactttagcgtgatccagtacaacggcggtctgaacgaccagaactccgccagtgacagcggggaggccaatctcgacctgcagtacatcgttggtgtcagctcgcccattccggtcaccgagttcagcaccggtggccggggtcttctcattccggacctgagccagcccgaccccaacgacaacagcaacgagccgtatctggaattcctgcagaatgtgttgaagatggaccaggataagctccctcaggtcatctccacctcctatggcgaggatgaacagaccattcccgaaaaatacgcgcgctcggtctgcaacctgtacgctcagctgggcagccgcggggtttcggtcattttctcctctggtgactccggtgttggcgcggcttgcttgaccaacgacggcaccaaccgcacgcacttccccccacagttccctgcggcctgcccctgggtgacctcggtgggtggcacgaccaagacccagcccgaggaggcggtgtacttttcgtcgggcggtttctccgacctgtgggagcgcccttcctggcaggattcggcggtcaagcgctatctcaagaagctgggccctcggtacaagggcctgtacaaccccaagggccgtgccttccccgatgttgctgcccaggccgagaactacgccgtgttcgacaagggggtgctgcaccagtttgacggaacctcgtgctcggctcccgcatttagcgctatcgtcgcattgctgaacgatgcgcgtctgcgcgctcacaagcccgtcatgggtttcctgaacccctggctgtatagcaaggccagcaagggtttcaacgatatcgtcaagggcggtagcaagggctgcgacggtcgcaaccgattcggaggtactcccaatggcagccctgtggtgccctatgccagctggaatgccactgacggctgggacccggccacgggtctagggactccggactttggcaagcttctgtctcttgctatgcggagatag

SEQ ID NO: 2 - DNA, 1797 bp, Artificial Sequence
  TRI039 synthetic gene
atgcagaccttcggtgcttttctcgtttccttcctcgccgccagcggcctggccgcggccgaggtctttgagaagctcagcgctgtcccccagggctggaagtacagccacacccctagcgaccgcgaccccatccgcctccagatcgccctcaagcagcacgacgtcgagggcttcgagactgccctccttgagatgagcgacccctaccaccccaactacggcaagcacttccagacccacgaagagatgaagcgcatgctcctgcccacccaagaggccgtcgagtctgtccgcggctggcttgagagcgccggcatcagcgacatcgaagaggacgccgactggatcaagttccgcaccaccgtcggcgtcgccaacgacctcctcgacgccgacttcaagtggtacgtcaacgaggtcggccacgtcgagcgcctccgaaccctcgcttacagcctccctcagagcgtcgccagccacgtcaacatggtccagcccaccacccgcttcggccagatcaagcctaaccgcgccaccatgcgaggccgccctgtccaggtcgacgccgacattctctctgccgccgtccaggccggcgacacctctacttgcgaccaggtcatcaccccccagtgcctcaaggacctctacaacatcggcgactacaaggccgaccccaacggcggcagcaaggtcgccttcgccagcttcctcgaagagtacgcccgctacgacgacctcgccaagttcgaggaaaagctcgccccctacgccatcggccagaacttcagcgtcatccagtacaacggcggcctcaacgaccagaacagcgccagcgatagcggcgaggccaacctcgacctccagtacatcgtcggcgtcagcagccccatccccgtcaccgagttttcgactggcggccgaggcctcctcatccccgatctcagccagcccgaccctaacgacaacagcaacgagccctaccttgagttcctccagaacgtcctcaagatggaccaggacaagctcccccaggtcatcagcaccagctacggcgaggacgagcagaccatccccgagaagtacgcccgcagcgtctgcaacctctacgcccagcttggctctcgcggcgtcagcgtcatcttcagctctggcgacagcggcgtcggcgctgcctgcctcactaacgacggcaccaaccgcacccacttcccgccccagtttcccgccgcttgcccttgggtcactagcgtcggcggcaccaccaagacccagcccgaggaagccgtctacttcagcagcggcggcttcagcgacctctgggagcgacctagctggcaggacagcgccgtcaagcgctacctcaagaagctcggccctcgctacaagggcctgtacaaccccaagggccgagccttccctgacgtcgccgctcaggccgagaactacgccgtctttgacaagggcgtcctccaccagttcgacggcaccagctgtagcgcccctgccttcagcgccatcgtcgccctgctcaacgacgcccgactccgcgcccacaagcccgtcatgggctttctcaacccctggctctacagcaaggccagcaagggcttcaacgacatcgtcaagggcggctccaagggctgcgacggccgcaaccgatttggcggcactcccaacggcagccccgtcgtcccttacgcctcttggaacgccaccgacggctgggaccctgctactggcctcggcacccccgacttcggcaagctcctctctctcgccatgcgccgctaa

SEQ ID NO: 3 - protein, 578 aa, Aspergillus fumigatus
EVFEKLSAVPQGWKYSHTPSDRDPIRLQIALKQHDVEGFETALLEMSDPYHPNYGKHFQTHEEMKRMLLPTQEAVESVRGWLESAGISDIEEDADWIKFRTTVGVANDLLDADFKWYVNEVGHVERLRTLAYSLPQSVASHVNMVQPTTRFGQIKPNRATMRGRPVQVDADILSAAVQAGDTSTCDQVITPQCLKDLYNIGDYKADPNGGSKVAFASFLEEYARYDDLAKFEEKLAPYAIGQNFSVIQYNGGLNDQNSASDSGEANLDLQYIVGVSSPIPVTEFSTGGRGLLIPDLSQPDPNDNSNEPYLEFLQNVLKMDQDKLPQVISTSYGEDEQTIPEKYARSVCNLYAQLGSRGVSVIFSSGDSGVGAACLTNDGTNRTHFPPQFPAACPWVTSVGGTTKTQPEEAVYFSSGGFSDLWERPSWQDSAVKRYLKKLGPRYKGLYNPKGRAFPDVAAQAENYAVFDKGVLHQFDGTSCSAPAFSAIVALLNDARLRAHKPVMGFLNPWLYSKASKGFNDIVKGGSKGCDGRNRFGGTPNGSPVVPYASWNATDGWDPATGLGTPDFGKLLSLAMRR

SEQ ID NO: 4 - protein, 392 aa, Aspergillus fumigatus
CDQVITPQCLKDLYNIGDYKADPNGGSKVAFASFLEEYARYDDLAKFEEKLAPYAIGQNFSVIQYNGGLNDQNSASDSGEANLDLQYIVGVSSPIPVTEFSTGGRGLLIPDLSQPDPNDNSNEPYLEFLQNVLKMDQDKLPQVISTSYGEDEQTIPEKYARSVCNLYAQLGSRGVSVIFSSGDSGVGAACLTNDGTNRTHFPPQFPAACPWVTSVGGTTKTQPEEAVYFSSGGFSDLWERPSWQDSAVKRYLKKLGPRYKGLYNPKGRAFPDVAAQAENYAVFDKGVLHQFDGTSCSAPAFSAIVALLNDARLRAHKPVMGFLNPWLYSKASKGFNDIVKGGSKGCDGRNRFGGTPNGSPVVPYASWNATDGWDPATGLGTPDFGKLLSLAM

SEQ ID NO: 5 - protein, 844 aa, Lactobacillus helveticus
MAVKRFYKTFHPEHYDLRINVNRKNKTINGTSTITGDVIENPVFINQKFMTIDSVKVDGKNVDFDVIEKDEAIKIKTGVTGKAVIEIAYSAPLTDTMMGIYPSYYELEGKKKQIIGTQFETTFARQAFPCVDEPEAKATFSLALKWDEQDGEVALANMPEVEVDKDGYHHFEETVRMSSYLVAFAFGELQSKTTHTKDGVLIGVYATKAHKPKELDFALDIAKRAIEFYEEFYQTKYPLPQSLQLALPDFSAGAMENWGLVTYREAYLLLDPDNTSLEMKKLVATVITHELAHQWFGDLVTMKWWDNLWLNESFANMMEYLSVDGLEPDWHIWEMFQTSEAASALNRDATDGVQPIQMEINDPADIDSVFDGAIVYAKGSRMLVMVRSLLGDDALRKGLKYYFDHHKFGNATGDDLWDALSTATDLDIGKIMHSWLKQPGYPVVNAFVAEDGHLKLTQKQFFIGEGEDKGRQWQIPLNANFDAPKIMSDKEIDLGNYKVLREEAGHPLRLNVGNNSHFIVEYDKTLLDDILSDVNELDPIDKLQLLQDLRLLAEGKQISYASIVPLLVKFADSKSSLVINALYTTAAKLRQFVEPESNEEKNLKKLYDLLSKDQVARLGWEVKPGESDEDVQIRPYELSASLYAENADSIKAAHQIFTENEDNLEALNADIRPYVLINEVKNFGNAELVDKLIKEYQRTADPSYKVDLRSAVTSTKDLAAIKAIVGDFENADVVKPQDLCDWYRGLLANHYGQQAAWDWIREDWDWLDKTVGGDMEFAKFITVTAGVFHTPERLKEFKEFFEPKINVPLLSREIKMDVKVIESKVNLIEAEKDAVNDAVAKAID

SEQ ID NO: 6 - protein, 525 aa, Aspergillus fumigatus
MRTAAASLTLAATCLFELASALMPRAPLIPAMKAKVALPSGNATFEQYIDHNNPGLGTFPQRYWYNPEFWAGPGSPVLLFTPGESDAADYDGFLTNKTIVGRFAEEIGGAVILLEHRYWGASSPYPELTTETLQYLTLEQSIADLVHFAKTVNLPFDEIHSSNADNAPWVMTGGSYSGALAAWTASIAPGTFWAYHASSAPVQAIYDFWQYFVPVVEGMPKNCSKDLNRVVEYIDHVYESGDIERQQEIKEMFGLGALKHFDDFAAAITNGPWLWQDMNFVSGYSRFYKFCDAVENVTPGAKSVPGPEGVGLEKALQGYASWFNSTYLPGSCAEYKYWTDKDAVDCYDSYETNSPIYTDKAVNNTSNKQWTWFLCNEPLFYWQDGAPKDESTIVSRIVSAEYWQRQCHAYFPEVNGYTFGSANGKTAEDVNKWTKGWDLTNTTRLIWANGQFDPWRDASVSSKTRPGGPLQSTEQAPVHVIPGGFHCSDQWLVYGEANAGVQKVIDEEVAQIKAWVAEYPKYRKP

(19) United States
(12) Patent Application Publication
(10) Pub. No.: US 2021/0120845 A1
(43) Pub. Date: Apr. 29, 2021

(54) METHOD FOR PRODUCING A PROTEIN HYDROLYSATE EMPLOYING AN ASPERGILLUS FUMIGATUS TRIPEPTIDYL PEPTIDASE

(71) Applicant: DUPONT NUTRITION BIOSCIENCES APS, Copenhagen (DK)

(72) Inventors: Karsten Matthias Kragh, Hoejbjerg (DK); Svend Haaning, Galten (DK); Ernst Meinjohanns, Frederiksberg C (DK); Peter Edvard Degn, Egå (DK); Steffan Yde Bak, Hadsten (DK); Thomas Eisele, Eichenau (DK)

(21) Appl. No.: 16/074,144

(22) PCT Filed: Feb. 21, 2017
(86) PCT No.: PCT/US2017/018686

§ 371 Date: Jul. 31, 2018

Related U.S. Application Data
(60) Provisional application No. 62/299,709, filed on Feb. 25, 2016.

(51) Int. Cl. A23K 20/189; A23K 10/14; C12N 9/48; A23K 20/147; A23L 2/66

(52) U.S. Cl. CPC A23K 20/189; A23K 10/14; C12N 9/485; C12Y 304/1401; A23L 2/66; C12Y 304/14009; A23K 20/147

(57) ABSTRACT

The present invention relates to compositions and methods for the production of a hydrolysate comprising at least one endoprotease and a tripeptidyl peptidase capable of cleaving tripeptides from the N-terminus a peptide and/or proteins having one or more of lysine, arginine or glycine in the P1 position wherein said tripeptidyl peptidase is capable of being used at a temperature between 45° C. and 70° C.

### FIELD OF THE INVENTION

The present invention relates to tripeptidyl peptidases for use in the preparation of hydrolysates and in foods comprising said tripeptidyl peptidases or hydrolysates.

### BACKGROUND

Proteases (synonymous with peptidases) are enzymes that are capable of cleaving peptide bonds between amino acids in substrate peptides, oligopeptides, and/or proteins.

Proteases are grouped into 7 families based on their catalytic reaction mechanism and the amino acid residue involved in the active site for catalysis. The serine proteases, aspartic acid proteases, cysteine proteases and metalloprotease are the 4 major families, whilst the threonine proteases, glutamic acid proteases and ungrouped proteases make up the remaining 3 families.

Proteases can be also generally subdivided into two broad groups based on their substrate-specificity. The first group is that of the endoproteases, which are proteolytic peptidases capable of cleaving internal peptide bonds of a peptide or protein substrate and tending to act away from the N-terminus or C-terminus. Examples of endoproteases include trypsin, chymotrypsin and pepsin. In contrast, the second group of proteases is the exopeptidases which cleave peptide bonds between amino acids located towards the C- or N-terminus of a protein or peptide substrate.

Certain enzymes of the exopeptidase group may have tripeptidyl peptidase activity. Such enzymes are therefore capable of cleaving 3 amino acid fragments (tripeptides) from the unsubstituted N-terminus of substrate peptides, oligopeptides and/or proteins. Tripeptidyl peptidases are known to cleave tripeptide sequences from the N-terminus of a substrate.

Both exopeptidases and endoproteases have many applications both in the food and feed industries and in the production of hydrolysates.

### SUMMARY OF THE INVENTION

According to a first aspect there is provided a method for the production of a hydrolysate comprising:

a) admixing at least one protein or a portion thereof with a tripeptidyl peptidase which:
i) comprises the amino acid sequence SEQ ID No. 3, SEQ ID No. 4 or a functional fragment thereof;
ii) comprises an amino acid having at least 70% identity to SEQ ID No. 3 or SEQ ID No. 4;
iii) is encoded by a nucleotide sequence comprising the sequence SEQ ID No. 1 or SEQ ID No. 2;
iv) is encoded by a nucleotide sequence which has at least about 70% identity to SEQ ID No. 1 or SEQ ID No. 2;
v) is encoded by a nucleotide sequence which hybridises to SEQ ID No. 1 or SEQ ID No. 2 under medium stringency conditions; or
vi) is encoded by a nucleotide sequence which differs from SEQ ID No. 1 or SEQ ID No. 2 due to degeneracy of the genetic code;

b) incubating at a temperature between 45° C. and 70° C., and

c) recovering the hydrolysate.

In a second aspect there is provided a reaction system comprising at least one protein or a portion thereof and a tripeptidyl peptidase which:
a) comprises the amino acid sequence SEQ ID No. 3, SEQ ID No. 4 or a functional fragment thereof;
b) comprises an amino acid having at least 70% identity to SEQ ID No. 3 or SEQ ID No. 4;
c) is encoded by a nucleotide sequence comprising the sequence SEQ ID No. 1 or SEQ ID No. 2;
d) is encoded by a nucleotide sequence which has at least about 70% identity to SEQ ID No. 1 or SEQ ID No. 2;
e) is encoded by a nucleotide sequence which hybridises to SEQ ID No. 1 or SEQ ID No. 2 under medium stringency conditions; or
f) is encoded by a nucleotide sequence which differs from SEQ ID No. 1 or SEQ ID No. 2 due to degeneracy of the genetic code;

wherein the reaction system is maintained at a temperature between 45° C. and 70° C. for a sufficient period of time to allow production of a hydrolysate.

In a third aspect there is provided a method for the expression of a tripeptidyl peptidase, wherein said method comprises:
a) transforming a Trichderma host cell with a nucleic acid or vector comprising
   i) the nucleotide sequence SEQ ID No. 1 or SEQ ID No. 2;
   ii) a nucleotide sequence which has at least about 70% identity to SEQ ID No. 1 or SEQ ID No. 2;
   iii) a nucleotide sequence which hybridises to SEQ ID No. 1 or SEQ ID No. 2 under medium stringency conditions; or
   iv) a nucleotide sequence which differs from SEQ ID No. 1 or SEQ ID No. 2 due to degeneracy of the genetic code;
b) expressing the nucleic acid sequence or vector of step a); and
c) obtaining the tripeptidyl peptidase or a fermentate comprising said tripeptidyl peptidase and optionally isolating and/or purifying and/or packaging.

In a fourth aspect there is provided the use of a tripeptidyl peptidase which:
i) comprises the amino acid sequence SEQ ID No. 3, SEQ ID No. 4 or a functional fragment thereof;
ii) comprises an amino acid having at least 70% identity to SEQ ID No. 3 or SEQ ID No. 4;
iii) is encoded by a nucleotide sequence comprising the sequence SEQ ID No. 1 or SEQ ID No. 2;
iv) is encoded by a nucleotide sequence which has at least about 70% identity to SEQ ID No. 1 or SEQ ID No. 2;
v) is encoded by a nucleotide sequence which hybridises to SEQ ID No. 1 or SEQ ID No. 2 under medium stringency conditions; or
vi) is encoded by a nucleotide sequence which differs from SEQ ID No. 1 or SEQ ID No. 2 due to degeneracy of the genetic code;
   in the manufacture of a hydrolysate at a temperature between 45° C. and 70° C.

In a fifth aspect there is provided a hydrolysate obtainable (preferably obtained) from any one of the method, reaction system or use provided herein.

In a sixth aspect there is provided a feed additive composition or food additive composition comprising the hydrolysate provided herein.

In a seventh aspect there is provided a method for producing a feedstuff or foodstuff comprising contacting a feed component or food component with the hydrolysate provided herein or a feed additive composition or feed additive composition as provided herein.

In a seventh aspect there is provided a feedstuff or foodstuff comprising a hydrolysate as provided herein or a feed additive composition or feed additive composition as provided herein.

In an eighth aspect there is provided a nonfood product comprising the hydrolysate provided herein, wherein the nonfood product is a cosmetic, a lotion, or a cleanser for use on human skin.

### BRIEF DESCRIPTION OF THE DRAWINGS

Embodiments of the invention will now be described, by way of example only, with reference to accompanying drawings, in which:

FIG. 1 shows a plasmid map of the expression vector pTTT-pyrG13-TRI039.

### DETAILED DESCRIPTION

A seminal finding is that the tripeptidyl peptidase as claimed herein can be used in a high temperature hydrolysis method, for example, between 45° C. and 70° C.

The inventors observed a significant and completely unexpected improvement when producing hydrolysates using this enzyme between 45° C. and 70° C. compared with hydrolysis at room temperature.

The inventors have shown for the first time that a tripeptidyl peptidase is highly advantageous for use in the preparation of hydrolysates at high temperatures (e.g. between 45° C. and 70° C.).

Alternatively or additionally, the hydrolysate produced using a tripeptidyl peptidase may have reduced immunogenicity in a subject predisposed to having an immune reaction to an untreated protein or portion thereof or may have reduced bitterness when compared to an untreated protein or hydrolysate.

Advantageously, a tripeptidyl peptidase taught for use in the present methods and compositions is capable of acting on a wide range of peptide and/or protein substrates and due to having such a broad substrate-specificity is not readily inhibited from cleaving substrates enriched in certain amino acids (e.g. lysine and/or arginine and/or glycine). The use of such a tripeptidyl peptidase therefore may efficiently and/or rapidly breakdown protein substrates (e.g. present in a substrate for preparation of a hydrolysate). r

Based on these findings, there is provided a method for the production of a hydrolysate comprising:
a) admixing at least one protein or a portion thereof with a tripeptidyl peptidase which:
   i) comprises the amino acid sequence SEQ ID No. 3, SEQ ID No. 4 or a functional fragment thereof;
   ii) comprises an amino acid having at least 70% identity to SEQ ID No. 3 or SEQ ID No. 4;
   iii) is encoded by a nucleotide sequence comprising the sequence SEQ ID No. 1 or SEQ ID No. 2;
   iv) is encoded by a nucleotide sequence which has at least about 70% identity to SEQ ID No. 1 or SEQ ID No. 2;
   v) is encoded by a nucleotide sequence which hybridises to SEQ ID No. 1 or SEQ ID No. 2 under medium stringency conditions; or
   vi) is encoded by a nucleotide sequence which differs from SEQ ID No. 1 or SEQ ID No. 2 due to degeneracy of the genetic code;
b) incubating at a temperature between 45° C. and 70° C., and
c) recovering the hydrolysate.

Suitably the method may comprise a further step of admixing the hydrolysate recovered in step (b) with at least one food or feed ingredient.

In one aspect, the tripeptidyl peptidase is used in combination with an endoprotease.

Suitably, the endoprotease and the tripeptidyl peptidase are added simultaneously.

Suitably, the protein or portion thereof may be admixed with the endoprotease before adding the tripeptidyl peptidase. Suitably, the protein or portion thereof may be admixed with the endoprotease before adding the tripeptidyl peptidase and one or more further protease(s) as detailed herein.

The term “admixing”, as used herein, refers to the mixing of one or more ingredients and/or enzymes where the one or more ingredients or enzymes are added in any order and in any combination. Suitably, admixing may relate to mixing one or more ingredients and/or enzymes simultaneously or sequentially.

In one embodiment, the one or more ingredients and/or enzymes may be mixed simultaneously.

In another embodiment, the one or more ingredients and/or enzymes may be mixed sequentially.

The term “recovering a hydrolysate”, as used herein, refers to the isolation of a hydrolysate. In some embodiments this may involve separating the hydrolysed matter from unhydrolyzed protein and/or peptide substrates. In other embodiments it may additionally or alternatively involve separating the hydrolysed matter away from a tripeptidyl peptidase used for preparing said hydrolysate. In one embodiment, the hydrolysate may comprise hydrolysed matter with a purity of at least 90%, more suitably at least 95%, and even more suitably at least 99%.

A tripeptidyl peptidase for use in the methods and/or uses described herein may be incubated with a substrate (e.g. a protein and/or peptide substrate) at a temperature of at least about 45° C. In other words the method may be carried out at a temperature of at least about 45° C.

Suitably, the tripeptidyl peptidase may be incubated with a substrate at a temperature of at least about 50° C.

In a preferred embodiment, the tripeptidyl peptidase may be incubated with a substrate at a temperature of at least about 55° C.

The tripeptidyl peptidase is incubated with a substrate (e.g. a protein and/or peptide substrate) at a temperature of between about 45° C. to about 70° C. In other words the method is carried out at a temperature of between about 45° C. to about 70° C.

Suitably, the tripeptidyl peptidase may be incubated with a substrate at a temperature of between about 45° C. to about 65° C.; more suitably at a temperature of between about 50° C. to about 65° C.

In a preferred embodiment, the tripeptidyl peptidase may be incubated with a substrate at a temperature of between about 50° C. to about 60° C.

In a preferred embodiment, the tripeptidyl peptidase may be incubated with a substrate at a temperature of between about 55° C. to about 60° C.

The term “tripeptidyl peptidase”, as used herein, relates to an exopeptidase which can cleave tripeptides from the N-terminus of a peptide, oligopeptide and/or protein substrate.

In one embodiment, the tripeptidyl peptidase is not an endoprotease.

In another embodiment, the tripeptidyl peptidase is not an enzyme which cleaves tetrapeptides from the N-terminus of a substrate.

In a further embodiment, the tripeptidyl peptidase is not an enzyme which cleaves dipeptides from the N-terminus of a substrate.

In a yet further embodiment, the tripeptidyl peptidase is not an enzyme which cleaves single amino acids from the N-terminus of a substrate.

In one embodiment, the tripeptidyl peptidase may comprise a catalytic triad of the amino acids serine, aspartate, and histidine.

In one embodiment, the tripeptidyl peptidase may be a thermostable tripeptidyl peptidase.

The term “thermostable”, as used herein, means that an enzyme retains its activity when heated to temperatures of up to about 70° C. In a preferred embodiment, “thermostable” means that an enzyme retains its activity when heated to about 65° C.; more suitably to about 60° C.

Advantageously, a thermostable tripeptidyl peptidase is less prone to being denatured and/or will retain its activity for a longer period of time when compared to a non-thermostable variant.

In one embodiment, the tripeptidyl peptidase has activity in a range of about pH 2 to about pH 7. Suitably, the tripeptidyl peptidase has activity in a range of about pH 4 to about pH 7 and even more suitably in a range of about pH 4.5 to about pH 6.5.

Suitably, the present method, in particular the hydrolysis step, may be carried out at a pH of between 2 to about 7.

In one embodiment, the present method, in particular the hydrolysis step may be carried out at a pH of between about 4 to about 7, for example4.5 to 6.5.

Using a tripeptidyl peptidase having activity in a pH range between about pH 4 to about pH 7 is advantageous as it allows the tripeptidyl peptidase to be used with one more endoproteases having activity in this pH range.

When a tripeptidyl peptidase having activity in a pH range between about pH 4 to about pH 7 is used, suitably it may be used in combination with a neutral or an alkaline endoprotease.

Advantageously this means that changing the pH of the reaction medium comprising the protein and/or peptide substrate for hydrolysate production is not necessary between enzyme treatments. In other words, it allows the tripeptidyl peptidase and the endoprotease to be added to a reaction simultaneously, which may make the process for producing the hydrolysate quicker and/or more efficient and/or more cost-effective. Moreover, this allows for a more efficient reaction as at lower pH values the substrate may precipitate out of solution and therefore not be cleaved.

Any suitable alkaline endoprotease may be used. Suitably, the alkaline endoprotease may be one or more selected from the group consisting of: a trypsin, a chymotrypsin, and a subtilisin.

In another embodiment, the tripeptidyl peptidase may have activity at an acidic pH (suitably, the tripeptidyl peptidase may have optimum activity at acidic pH). The tripeptidyl peptidase may have activity at a pH of less than about pH 6, more suitably less than about pH 5. Preferably, the tripeptidyl peptidase may have activity at a pH of between about 2.5 to about pH 4.0, more suitably at between about 3.0 to about 3.3.

Suitably, the present method, in particular the hydrolysis step, may be carried out at a pH of between 2 to about 4, e.g. 3 to 3.3.

A tripeptidyl peptidase having activity at an acidic pH can be used in combination with an acid endoprotease and advantageously does not require the pH of the reaction medium comprising the protein and/or peptide substrate for hydrolysate production to be changed between enzyme treatments. In other words, it allows the tripeptidyl peptidase and the endoprotease to be added to a reaction simultaneously, which may make the process for producing the hydrolysate quicker and/or more efficient and/or more cost-effective.

At least one endoprotease may be used in combination with the tripeptidyl peptidase for any of the applications herein. For example, at least one endoprotease may be comprised in the composition and/or food additive composition and/or non-food product.

The term “endoprotease”, as used herein, is synonymous with the term “endopeptidase” and refers to an enzyme which is a proteolytic peptidase capable of cleaving internal peptide bonds of a peptide or protein substrate (e.g. not located towards the C or N-terminus of the peptide or protein substrate). Such endoproteases may be defined as ones that tend to act away from the N-terminus or C-terminus.

In one embodiment, the endoprotease may be one or more selected from the group consisting of: a serine protease, an aspartic acid protease, a cysteine protease, a metalloprotease, a threonine protease, a glutamic acid protease, and a protease selected from the family of ungrouped proteases.

In one embodiment, the endoprotease may be one or more selected from the group consisting of: an acid fungal protease, a subtilisin, a chymotrypsin, a trypsin, and a pepsin or from the group of commercial protease products such as Alphalase® AFP, Alphalase® FP2, Alphalase® NP, FoodPro® Alkaline Protease, FoodPro® PXT, FoodPro® PBR , FoodPro® 30L, FoodPro® PHT, and FoodPro® 51 FP.

In one embodiment, the endoprotease may be an acid endoprotease. Suitably, the endoprotease may be an acid fungal protease.

Advantageously, the use of an endoprotease in combination with a tripeptidyl peptidase can increase the efficiency of substrate cleavage. Without wishing to be bound by theory, it is believed that an endoprotease is capable of cleaving a peptide and/or protein substrate at multiple regions away from the C or N-terminus, thereby producing more N-terminal ends for the tripeptidyl peptidase to use as a substrate, thereby advantageously increasing reaction efficiency and/or reducing reaction times.

### Reaction System

A reaction system is also provided comprising at least one protein or a portion thereof and a tripeptidyl peptidase which: i) comprises the amino acid sequence SEQ ID No. 3, SEQ ID No. 4 or a functional fragment thereof; ii) comprises an amino acid having at least 70% identity to SEQ ID No. 3 or SEQ ID No. 4; iii) is encoded by a nucleotide sequence comprising the sequence SEQ ID No. 1 or SEQ ID No. 2; iv) is encoded by a nucleotide sequence which has at least about 70% identity to SEQ ID No. 1 or SEQ ID No. 2; v) is encoded by a nucleotide sequence which hybridises to SEQ ID No. 1 or SEQ ID No. 2 under medium stringency conditions; or vi) is encoded by a nucleotide sequence which differs from SEQ ID No. 1 or SEQ ID No. 2 due to degeneracy of the genetic code; wherein the reaction system is maintained at a temperature between 45° C. and 70° C. for a sufficient period of time to allow production of a hydrolysate.

In one embodiment, the reaction system temperature is maintained between 50° C. and 65° C. In a further embodiment, the temperature is maintained between 55° C. and 65° C.

In one embodiment, the reaction system further comprises an endoprotease. The endoprotease may be active at a similar pH range as the tripeptidyl peptidase. In one embodiment, the endoprotease is an acid endoprotease. In another embodiment the endoprotease may be an alkaline endoprotease, preferably selected from one or more of: a trypsin or a chymotrypsin.

The at least one protein or portion thereof in the reaction system may be an animal protein or a plant protein, preferably wherein the protein is one or more of a gliadin, a beta-casein, a beta-lactoglobulin or an immunogenic fragment of a gliadin, a beta-casein, a beta-lactoglobulin, whey protein, fish protein, meat protein, egg protein, soy protein, a hordein or grain protein.

### Hydrolysates

A hydrolysate is provided herein obtainable (e.g. obtained) by a method of the invention. Suitably, such a hydrolysate may be enriched in tripeptides.

The term “hydrolysate”, as used herein, has its usual meaning in the art and refers to a product resulting from the treatment of a protein or portion thereof with a tripeptidyl peptidase. The extent of proteolytic cleavage of the protein or portion thereof can range from minimal (e.g., cleavage of a single peptide bond on a single protein) to extensive depending on, for example, the conditions of the treatment, such as the length of the treatment, the temperature, the concentration of the protein, and the purity, concentration, and activity of the tripeptidyl peptidase.

A “hydrolysate” typically comprises a mixture of short peptides obtainable by cleaving a peptide and/or protein substrate with at least one protease (suitably a tripeptidyl peptidase). Suitably, such a hydrolysate may be substantially enriched in tripeptides.

The term “substantially enriched in tripeptides”, as used herein, means that of the total peptide concentration measured by any method known in the art (e.g., liquid chromatography-mass spectrometry (LC-MS)) at least about 20%, suitably at least about 30%, of those peptides are tripeptides. Suitably, at least about 40% of those peptides are tripeptides, more suitably at least about 50%.

In one embodiment, the term “substantially enriched in tripeptides” means that of the total peptide concentration measured by any method known in the art (for example, liquid chromatography-mass spectrometry (LC-MS)) at least about 70% of those peptides are tripeptides.

In one embodiment, the hydrolysate comprises less than about 20%, suitably less than about 10% of the full-length starting substrate (e.g., at least one protein). Suitably, the hydrolysate may comprise less than about 5%, more suitably less than about 1% of the full-length starting substrate (e.g., at least one protein).

In some embodiments, the hydrolysate may comprise no, or substantially no, full-length starting substrate.

The term “substantially no”, as used in this context, may mean less than about 0.5%, suitably less than about 0.1% of full-length starting substrate.

Where an endoprotease, tripeptidyl peptidase, and aminopeptidase have been used in the manufacture of a hydrolysate, it is believed that such a hydrolysate will be enriched in single amino acids, dipeptides, and tripeptides.

In one embodiment, the single amino acids, dipeptides, and tripeptides present in such a hydrolysate may be quantified in terms of molarity of each stated. In one embodiment, the molar ratio of single amino acids, dipeptides, and tripeptides in a hydrolysate is at least about 20% single amino acids to at least about 10% dipeptides to at least about 10% tripeptides.

In another embodiment, the molar ratio of single amino acids, dipeptides, and tripeptides in a hydrolysate may be at least about 10% single amino acids to at least about 20% dipeptides to at least about 20% tripeptides.

The hydrolysate obtainable according to the present methods or for use in any of the applications taught herein may have a reduced immunogenicity in a subject predisposed to having an immune response to the at least one protein or a portion thereof that formed the substrate for digestion for the production of the hydrolysate.

The hydrolysate is produced by admixing at least one protein or a portion thereof with a tripeptidyl peptidase.

The protein or portion thereof used as the substrate in manufacture of the hydrolysate may be an animal protein or a plant protein (for example, a vegetable protein).

Suitably, the protein or portion thereof may be one or more selected from the group consisting of: a gliadin, a beta-casein, a beta-lactoglobulin or an immunogenic fragment of a gliadin, a beta-casein, a beta-lactoglobulin, glycinin, beta-conglycinin, cruciferin, napin, collagen, whey protein, fish protein, meat protein, egg protein, soy protein, a hordein, and a grain protein.

In one preferred embodiment, the protein or portion thereof is a plant protein, a milk based protein, an egg protein or any combination thereof.

In one preferred embodiment, the protein or portion thereof is a plant protein, preferably wherein the protein is one or more of a gliadin, an immunogenic fragment of a gliadin, a grain protein, gluten, and a soy protein.

In one preferred embodiment, the protein or portion thereof is a milk-based protein, preferably wherein the protein is one or more of a casein, e.g., beta-casein; a lactoglobulin, e.g., beta-lactoglobulin and a whey protein.

In one preferred embodiment, the protein or portion thereof is an egg protein.

The protein or portion thereof may be comprised in corn, soybean meal, corn dried distillers grains with solubles (DDGS), wheat, wheat proteins (including gluten), wheat by-products, wheat bran, corn by-products including corn gluten meal, barley, oat, rye, triticale, full fat soy, animal by-product meals, an alcohol-soluble protein (preferably a zein (e.g. a maize zein maize) and/or a kafirin (e.g. from sorghum)), a protein from oil seeds (preferably from soybean seed proteins, sun flower seed proteins, rapeseed proteins, canola seed proteins or combinations thereof) or any combination thereof.

Suitably, the protein or portion thereof may be one or more selected from the group consisting of: a wheat protein, portions of a wheat protein, a dairy protein, and portions of a dairy protein.

The wheat protein or portion thereof may be obtainable (or obtained from) from wheat, wheat products (e.g., wheat flour), wheat by-products, and/or wheat bran. Suitably, the wheat protein may be one or more selected from the group consisting of: gliadin, portions of gliadin, gluten, and portions of gluten.

The dairy protein or a portion thereof may be milk protein. Suitably, the milk protein may be one or more selected from the group consisting of: a beta-casein, a beta-lactoglobulin, and a whey protein.

In one embodiment, the protein or portion thereof may be a protein-meal. In one embodiment, this is a protein-meal from fish or a protein-meal from another non-human animal (for example, a non-human mammal or bird).

In some embodiments, the protein or a portion thereof may be an animal by-product. Such by-products may include tissues from animal production and processing which are not utilized in human food and are processed into an array of protein meals used in animal feeds and pet food. In one embodiment, the animal protein by-products may be meat and bone meal, meat meal, blood meal, poultry by-product meal, poultry meal, feather meal, and fish meal.

In another embodiment, the protein or a portion thereof may be a microbial protein. Microbial proteins, for example yeast extracts, are typically made by extracting the cell contents from microbial cultures; they may be used as food additives or flavourings, or as nutrients for microbial culture media.

In yet further embodiments, the protein or a portion thereof may be an invertebrate protein, suitably an insect protein. Insects/invertebrates possess enormous biodiversity and represent a large biomass (95% of the animal kingdom), and thus offer alternative protein sources.

As used herein, the term “portion thereof” in relation to a protein or portion thereof used for the manufacture of a hydrolysate may be an immunogenic fragment of a protein. As used herein, an “immunogenic fragment” is any portion that is capable of eliciting an immune response in a sensitive individual. The “immunogenic fragment” or “portion thereof” is a region of a full-length protein comprising or consisting of at least 10 amino acids, suitably at least 20 amino acids, and more suitably at least 30 amino acids.

In some embodiments, the immunogenic fragment or portion thereof may be at least about 50 amino acids, suitably at least about 100 amino acids, and more suitably at least about 200 amino acids.

As used herein, the term “milk protein” encompasses any naturally-occurring protein in the normal secretion of the mammary gland of a postpartum female mammal, or products derived therefrom, such as fractions thereof, or components made therefrom or thereof. The milk can be from any mammalian species including but not limited to cow, goat, sheep, buffalo, yak, camel, llama, alpaca, and human. Milk proteins from those mammals whose milk is used commercially or widely in various cultures and countries are preferred. It is to be noted that “milk protein” as used herein encompasses both the singular and the plural of the word “protein”, thus, the term “milk protein” may refer to a single protein, or any mixture of one or more proteins, except as otherwise indicated.

As used herein, the term “whey protein” encompasses any protein found in any amount in “whey”; the liquid by-product of cheese making that is separated from the curd. The whey resulting from the production of many cheeses is particularly low in micellar milk proteins, such as caseins, but relatively enriched in soluble proteins such as alpha lactalbumin and beta-lactoglobulin. As with “milk protein” above, the term “whey protein” as used herein encompasses both the singular and the plural of the word “protein”, thus, the term “whey protein” also may refer to a single protein, or any mixture of one or more whey proteins, except as otherwise indicated. It will be understood by the skilled artisan that whey proteins are in fact a subclass of milk proteins, and thus the term “milk protein” may include one or more whey proteins, except as otherwise indicated herein. Whey compositions may include, for example, milk, cream, and cheese whey. Whey derived from any cheese type may be used. Whey protein may be derived from any methods such as filtration, dialysis, evaporation, and reverse osmosis of cheese whey, or by any other process which results in the proteins typically described as “whey proteins”.

In a preferred embodiment, the hydrolysate obtainable (e.g., obtained) by the present method(s) may be a milk protein hydrolysate, a wheat protein hydrolysate (e.g., a gliadin and/or gluten hydrolysate), a soy protein hydrolysate or any combination thereof.

In another embodiment, a use of at least one tripeptidyl peptidase or fermentate in the manufacture of a hydrolysate at a temperature between 45° C. and 70° C. is also provided comprising a tripeptidyl peptidase which (a) comprises the amino acid sequence SEQ ID No. 3 or SEQ ID No. 4, or a functional fragment thereof; (b) comprises an amino acid having at least 70% identity to SEQ ID No. 3 or SEQ ID No. 4; (c) is encoded by a nucleotide sequence comprising the sequence SEQ ID No. 1 or SEQ ID No. 2; (d) is encoded by a nucleotide sequence which has at least about 70% identity to SEQ ID No. 1 or SEQ ID No. 2; (e) is encoded by a nucleotide sequence which hybridises to SEQ ID No. 1or SEQ ID No. 2 under medium stringency conditions; or (f) is encoded by a nucleotide sequence which differs from SEQ ID No. 1 or SEQ ID No. 2 due to degeneracy of the genetic code.

In one embodiment, the use of the tripeptidyl peptidase or fermentate comprising a tripeptidyl peptidase is for reducing the immunogenicity of the hydrolysate in a subject predisposed to having an immune reaction to an untreated protein or portion thereof.

As used herein, “reduced immunogenicity” refers to any reduction, decrease, or amelioration of a measurable immunological response. The measurement of such response may be assessed in vitro or in vivo. For example, the response may be measured directly or indirectly in a biological sample comprising tissue, cells, or fluid, or the like, or any combination thereof from an individual or it may be assessed in the individual, either directly or indirectly. While in various embodiments provided herein, any mathematical decrease (or reduction) in such response, whether measured in vitro or in vivo, will suffice, it is preferred that the decrease be a more substantial one. Of course, the skilled artisan will appreciate that biological data such as a measurement of an immunological response, are subject to potentially large variation within an individual, and from individual to individual. For example where the response is in a “sensitive” individual, the immune response is preferably substantially reduced (e.g., by at least about 50%, 60%, or 70%, or even about 80% or more). More preferably, only small or minimal differences are seen in such the sensitive individual with the protein hydrolysates described herein, as compared to a measure of the immunological response from an individual who is not sensitive to one or more proteins or portions thereof. This is particularly preferable where the immunological response is deemed adverse, for example an allergic response. In such cases the decrease in the sensitive individual's immunological response (or measure thereof) may be at least about 85% to about 90%, more preferably about 90% to about 95%, or even more. In some cases, a sensitive individual's response to the protein hydrolysates described herein is not significantly different, statistically, from the response of an individual who is not sensitive. In yet other cases, the reduction in immunological response may be many-fold over that seen with the unhydrolyzed protein in a “sensitive” individual. For example, there may be about a 10-fold to 100-fold or even 1000-fold reduction in response. More preferably reductions of about 1000-fold to 10,000-fold or even 100,000-fold or greater reduction in a measurement of an immunological response from an individual consuming or exposed to the protein hydrolysate compositions as disclosed herein, as compared to that individual's response to the unmodified proteins.

As used herein, a “sensitive” individual is an individual predisposed to having an immune response or reaction to the protein in an unhydrolyzed form. Such immune response or reaction as a direct or indirect result of the consumption of, or exposure to, for example one or more protein or portion thereof, is a measure of the immunogenicity of those proteins. Such proteins will demonstrate little to no immunogenicity in an individual who is not predisposed to having such an immune response to the protein, such an individual is sometimes referred to herein as “insensitive” or “not sensitive” to the one or more proteins or portions thereof. Preferably, such an individual will not have a significant immune reaction (immunological response) to either the exposure to or consumption of the protein.

Sensitive individuals having a reaction to wheat proteins (in particular gluten and/or gliadin) may present with symptoms of coeliac disease (e.g., celiac sprue). Symptoms include pain and discomfort in the digestive tract, chronic constipation and diarrhea, failure to thrive (in children), anemia and fatigue, but these may be absent, and symptoms in other organ systems have been described. Vitamin deficiencies are often noted in people with coeliac disease owing to the reduced ability of the small intestine to properly absorb nutrients from food. Without wishing to be bound by theory it is believed that upon exposure to gliadin that the enzyme tissue transglutaminase modifies the gliadin resulting in cross-reaction of the immune system with the bowel tissue causing an inflammatory reaction in the affected individual.

Sensitive individuals having a reaction to milk proteins may present with symptoms of a milk allergy which is caused by an adverse immune reaction to one or more of the proteins or immunogenic fragments thereof in the milk. The disorder can be either antibody-mediated or non-antibody-mediated. Antibody-mediated milk allergies are typically rapid in onset and may result in the individual displaying gastrointestinal, dermatological and/or respiratory symptoms. Such symptoms may further manifest as: skin rashes, hives, vomiting, and gastric distress, such as diarrhea, rhinitis, stomach pain, wheezing, or anaphylactic reactions. Non-antibody-mediated is typically believed to be mediated by T-lymphocytes and not caused by antibodies. Symptoms of this form are typically gastrointestinal and dermatological.

Other sensitive individuals may have a reaction to soy which results in a range of symptoms, the most severe being anaphylaxis.

The hydrolysates and/or food and/or feed and/or comprising such hydrolysates or compositions may be particularly suitable for administering to a subject suffering from coeliac disease, a milk protein allergy and/or a soy protein allergy.

Advantageously, the endoprotease in combination with a tripeptidyl peptidase is capable of cleaving protein substrates associated with causing an immune response in sensitive individuals suffering from a disease, such as a milk protein allergy and/or a soy protein allergy.

In another aspect, the use of the at least one tripeptidyl peptidase or fermentate comprising a tripeptidyl peptidase is for reducing bitterness of the hydrolysate.

When the protein substrate or portion thereof for hydrolysate production is rich in hydrophobic L-amino acids the protein hydrolysate may have a bitter taste. Without wishing to be bound by theory, it is believed that the bitterness of a peptide is dependent on its peptide length and the average hydrophobicity of the L-amino acid residues therein.

The “reduced bitterness” of a hydrolysate can be measured objectively using a tasting panel of individuals who are asked to rate the bitterness of a hydrolysate. A bitterness index can be used to rate the bitterness of the hydrolysate. For example, a bitterness index can be used that rates substances relative to quinine which is given a reference index of 1; alternatively a bitterness index may be used having a scale from 0 (not bitter) to 10 (bitter). Suitably, any tasting of hydrolysates may be done using the appropriate controls, such as blind testing. Additionally or alternatively, the cleavage of known bitter peptides may be monitored via LC-MS or other suitable techniques known in the art.

The tripeptidyl peptidase may be used to cleave bitter peptides.

In one embodiment, debittered hydrolysates may be used in the preparation of food and or foodstuffs.

### Activity and Assays

In one embodiment, the tripeptidyl peptidase is an exopeptidase. In other words it predominantly has exopeptidase activity.

The term “exopeptidase” activity, as used herein, means that the tripeptidyl peptidase is capable of cleaving tri-peptides from the N-terminus of a substrate, such as a protein, and/or peptide substrate.

The term “predominantly has exopeptidase activity”, as used herein, means that the tripeptidyl peptidase has no or substantially no endoprotease activity.

As used herein, “substantially no endoprotease activity” means that the tripeptidyl peptidase has less than about 100 U endoprotease activity in the “Endoprotease Assay” taught herein when compared to 1000 nkat of exopeptidase activity in the “Exopeptidase Broad-Specificity Assay (EBSA)” taught herein. Suitably, “substantially no endoprotease activity” means that the tripeptidyl peptidase has less than about 1000 endoprotease activity in the “Endoprotease Assay” taught herein when compared to 1000nkat of exopeptidase activity in the “Exopeptidase Broad-Specificity Assay” taught herein.

Preferably, the tripeptidyl peptidase may have less than about 10U endoprotease activity in the “Endoprotease Assay” taught herein when compared to 1000 nkat of exopeptidase activity in the “Exopeptidase Broad-Specificity Assay” taught herein, more preferably less than about 1U endoprotease activity in the “Endoprotease Assay” taught herein when compared to 1000 nkat of exopeptidase activity in the “Exopeptidase Broad-Specificity Assay” taught herein. Even more preferably the tripeptidyl peptidase may have less than about 0.1 U endoprotease activity in the “Endoprotease Assay” taught herein when compared to 1000 nkat of exopeptidase activity in the “Exopeptidase Broad-Specificity Assay” taught herein.

### “Endoprotease Assay”

### Azocasein Assay for Endoprotease Activity

A modified version of the endoprotease assay described by Iversen and Jorgensen, 1995 (Biotechnology Techniques 9, 573-576) is used. An enzyme sample of 50 μL is added to 250 μL of azocasein (0.25% w/v; from Sigma) in 4 times diluted Mcllvaine buffer, pH 5 and incubated for 15 min at 40° C. with shaking (800 rpm). The reaction is terminated by adding 50 μL of 2 M trichloroacetic acid (TCA) (from Sigma Aldrich, Denmark) and centrifugation for 5 min at 20,000×g. To a 195 μL sample of the supernatant, 65 μL of 1 M NaOH is added and absorbance at 450 nm is measured. One unit of endoprotease activity is defined as the amount which yields an increase in absorbance of 0.1 in 15 min at 40° C. at 450 nm.

### Amino Acid and Nucleotide Sequences

The tripeptidyl peptidase may be obtainable (e.g., obtained) from any source so long as it has the activity described herein.

In one embodiment, the tripeptidyl peptidase may be obtainable (e.g., obtained) from Aspergillus.

Suitably, the tripeptidyl peptidase may be obtainable (e.g., obtained) from Aspergillus fumigatus, more suitably from Aspergillus fumigatus AF293.

| SEQ | | | |
|---|---|---|---|
| ID | | | |
| No.: | Description | Sequence | Origin |
| 1 | TRI039 | ATGTTTTCGTCGCTCTTGAACCGTGGAGCTTTGCTCGCGGTTGTTTCTC | Aspergillus |
| | Genomic | TCTTGTCCTCTTCCGTTGCTGCCGAGGTTTTTGAGAAGCTGTCCGCGGT | fumigatus |
| | sequence | GCCACAGGGTTTGTTCTCCCGACCCCCCGCCTCTTACGTCGTGACTGAC | AF293 |
| | CDS | GAGAACAGGATGGAAATACTCCCACACCCCTAGTGACCGCGATCCCATT | |
| | | CGCCTCCAGATTGCCCTGAAGCAACATGATGTCGAAGGTTTTGAGACCG | |
| | | CCCTCCTGGAAATGTCCGATCCCTACCACCCAAACTATGGCAAGCACTT | |
| | | TCAAACTCACGAGGAGATGAAGCGGATGCTGCTGCCCACCCAGGAGGCG | |
| | | GTCGAGTCCGTCCGCGGCTGGCTGGAGTCCGCTGGAATCTCGGATATCG | |
| | | AGGAGGATGCAGACTGGATCAAGTTCCGCACAACCGTTGGCGTGGCCAA | |
| | | TGACCTGCTGGACGCCGACTTCAAGTGGTACGTGAACGAGGTGGGCCAC | |
| | | GTTGAGCGCCTGAGGACCCTGGCATACTCGCTCCCGCAGTCGGTCGCGT | |
| | | CGCACGTCAACATGGTCCAGCCCACCACGCGGTTCGGACAGATCAAGCC | |
| | | CAACCGGGCGACCATGCGCGGTCGGCCCGTGCAGGTGGATGCGGACATC | |
| | | CTGTCCGCGGCCGTTCAAGCCGGCGACACCTCCACTTGCGATCAGGTCA | |
| | | TCACCCCTCAGTGCCTCAAGGATCTGTACAATATCGGCGACTACAAGGC | |
| | | CGACCCCAACGGGGGCAGCAAGGTCGCGTTTGCCAGTTTCCTGGAGGAA | |
| | | TACGCCCGCTACGACGATCTGGCCAAGTTCGAGGAGAAGCTGGCCCCGT | |
| | | ACGCCATTGGACAGAACTTTAGCGTGATCCAGTACAACGGCGGTCTGAA | |
| | | CGACCAGAACTCCGCCAGTGACAGCGGGGAGGCCAATCTCGACCTGCAG | |
| | | TACATCGTTGGTGTCAGCTCGCCCATTCCGGTCACCGAGTTCAGCACCG | |
| | | GTGGCCGGGGTCTTCTCATTCCGGACCTGAGCCAGCCCGACCCCAACGA | |
| | | CAACAGCAACGAGCCGTATCTGGAATTCCTGCAGAATGTGTTGAAGATG | |
| | | GACCAGGATAAGCTCCCTCAGGTCATCTCCACCTCCTATGGCGAGGATG | |
| | | AACAGACCATTCCCGAAAAATACGCGCGCTCGGTCTGCAACCTGTACGC | |
| | | TCAGCTGGGCAGCCGCGGGGTTTCGGTCATTTTCTCCTCTGGTGACTCC | |
| | | GGTGTTGGCGCGGCTTGCTTGACCAACGACGGCACCAACCGCACGCACT | |
| | | TCCCCCCACAGTTCCCTGCGGCCTGCCCCTGGGTGACCTCGGTGGGTGG | |
| | | CACGACCAAGACCCAGCCCGAGGAGGCGGTGTACTTTTCGTCGGGCGGT | |
| | | TTCTCCGACCTGTGGGAGCGCCCTTCCTGGCAGGATTCGGCGGTCAAGC | |
| | | GCTATCTCAAGAAGCTGGGCCCTCGGTACAAGGGCCTGTACAACCCCAA | |
| | | GGGCCGTGCCTTCCCCGATGTTGCTGCCCAGGCCGAGAACTACGCCGTG | |
| | | TTCGACAAGGGGGTGCTGCACCAGTTTGACGGAACCTCGTGCTCGGCTC | |
| | | CCGCATTTAGCGCTATCGTCGCATTGCTGAACGATGCGCGTCTGCGCGC | |
| | | TCACAAGCCCGTCATGGGTTTCCTGAACCCCTGGCTGiATAGCAAGGCC | |
| | | AGCAAGGGTTTCAACGATATCGTCAAGGGCGGTAGCAAGGGCiGCGACG | |
| | | GTCGCAACCGATTCGGAGGTACTCCCAATGGCAGCCCTGTGGTGCCCTA | |
| | | TGCCAGCTGGAATGCCACTGACGGCTGGGACCCGGCCACGGGTCTAGGG | |
| | | ACTCCGGACTTTGGCAAGCrTCTGTCTCTTGCTATGCGGAGATAG | |
| 2 | TRI039 | ATGCAGACCTTCGGTGCTTTTCTCGTTTCCTTCCTCGCCGCCAGCGGCC | Aspergillus |
| | Synthetic | TGGCCGCGGCCGAGGTCTTTGAGAAGCTCAGCGCTGTCCCCCAGGGCTG | fumigatus |
| | Gene | GAAGTACAGCCACACCCCTAGCGACCGCGACCCCATCCGCCTCCAGATC | AF293 |
| | optimized for | GCCCTCAAGCAGCACGACGTCGAGGGCTTCGAGACTGCCCTCCTTGAGA | |
| | expression in | TGAGCGACCCCTACCACCCCAACTACGGCAAGCACTTCCAGACCCACGA | |
| | Trichoderma | AGAGATGAAGCGCATGCTCCTGCCCACCCAAGAGGCCGTCGAGTCTGTC | |
| | with | CGCGGCTGGCTTGAGAGCGCCGGCATCAGCGACATCGAAGAGGACGCCG | |
| | Trichoderma | ACTGGATCAAGTTCCGCACCACCGTCGGCGTCGCCAACGACCTCCTCGA | |
| | signal | CGCCGACTTCAAGTGGTACGTCAACGAGGTCGGCCACGTCGAGCGCCTC | |
| | sequence | CGAACCCTCGCTTACAGCCTCCCTCAGAGCGTCGCCAGCCACGTCAACA | |
| | underlined | TGGTCCAGCCCACCACCCGCTTCGGCCAGATCAAGCCTAACCGCGCCAC | |
| | | CATGCGAGGCCGCCCTGTCCAGGTCGACGCCGACATTCTCTCTGCCGCC | |
| | | GTCCAGGCCGGCGACACCTCTACTTGCGACCAGGTCATCACCCCCCAGT | |
| | | GCCTCAAGGACCTCTACAACATCGGCGACIACAAGGCCGACCCCAACGG | |
| | | CGGCAGCAAGGTCGCCTTCGCCAGCTTCCTCGAAGAGTACGCCCGCTAC | |
| | | GACGACCTCGCCAAGTTCGAGGAAAAGCTCGCCCCCTACGCCATCGGCC | |
| | | AGAACTTCAGCGTCATCCAGTACAACGGCGGCCTCAACGACCAGAACAG | |
| | | CGCCAGCGATAGCGGCGAGGCCAACCTCGACCTCCAGTACATCGTCGGC | |
| | | GTCAGCAGCCCCATCCCCGTCACCGAGTTTTCGACTGGCGGCCGAGGCC | |
| | | TCCTCATCCCCGATCTCAGCCAGCCCGACCCTAACGACAACAGCAACGA | |
| | | GCCCTACCTTGAGTTCCTCCAGAACGTCCTCAAGATGGACCAGGACAAG | |
| | | CTCCCCCAGGTCATCAGCACCAGCTACGGCGAGGACGAGCAGACCATCC | |
| | | CCGAGAAGTACGCCCGCAGCGTCTGCAACCTCTACGCCCAGCTTGGCTC | |
| | | TCGCGGCGTCAGCGTCATCTTCAGCTCTGGCGACAGCGGCGTCGGCGCT | |
| | | GCCTGCCTCACTAACGACGGCACCAACCGCACCCACTTCCCGCCCCAGT | |
| | | TTCCCGCCGCTTGCCCTTGGGTCACTAGCGTCGGCGGCACCACCAAGAC | |
| | | CCAGCCCGAGGAAGCCGTCTACTTCAGCAGCGGCGGCTTCAGCGACCTC | |
| | | TGGGAGCGACCTAGCTGGCAGGACAGCGCCGTCAAGCGCTACCTCAAGA | |
| | | AGCTCGGCCCTCGCTACAAGGGCCTGTACAACCCCAAGGGCCGAGCCTT | |
| | | CCCTGACGTCGCCGCTCAGGCCGAGAACTACGCCGTCTTTGACAAGGGC | |
| | | GTCCTCCACCAGTTCGACGGCACCAGCTGTAGCGCCCCTGCCTTCAGCG | |
| | | CCATCGTCGCCCTGCTCAACGACGCCCGACTCCGCGCCCACAAGCCCGT | |
| | | CATGGGCTTTCTCAACCCCTGGCTCTACAGCAAGGCCAGCAAGGGCTTC | |
| | | AACGACATCGTCAAGGGCGGCTCCAAGGGCTGCGACGGCCGCAACCGAT | |
| | | TTGGCGGCACTCCCAACGGCAGCCCCGTCGTCCCTTACGCCTCTTGGAA | |
| | | CGCCACCGACGGCTGGGACCCTGCTACTGGCCTCGGCACCCCCGACTTC | |
| | | GGCAAGCTCCTCTCTCTCGCCATGCGCCGCTAA | |
| 3 | TRI039 | EVFEKLSAVPQGWKYSHTPSDRDPIRLQIALKQHDVEGFETALLEMSDP | Aspergillus |
| | pre_pro | YHPNYGKHFQTHEEMKRMLLPTQEAVESVRGWLESAGISDIEEDADWIK | fumigatus |
| | amino acid | FRTTVGVANDLLDADFKWYVNEVGHVERLRTLAYSLPQSVASHVNMVQP | AF293 |
| | sesquence | TTRFGQTKPNRATMRGRPVQVDADTLSAAVQAGDTSTCDQVTTPQCLKD | |
| | | LYNIGDYKADPNGGSKVAFASFLEEYARYDDLAKFEEKLAPYATGQNFS | |
| | | VTQYNGGLNDQNSASDSGEANLDLQYTVGVSSPIPVTEFSTGGRGLLIP | |
| | | DLSQPDPNDNSNEPYLEFLQNVLKMDQDKLPQVTSTSYGEDEQTTPEKY | |
| | | ARSVCNLYAQLGSRGVSVTFSSGDSGVGAACLTNDGTNRTHFPPQFPAA | |
| | | CPWVTSVGGTTKTQPEEAVYFSSGGFSDLWERPSWQDSAVKRYLKKLGP | |
| | | RYKGLYNPKGRAFPDVAAQAENYAVFDKGVLHQFDGTSCSAPAFSATVA | |
| | | LLNDARLRAHKPVMGFLNPWLYSKASKGFNDTVKGGSKGCDGRNRFGGT | |
| | | PNGSPWPYASWNATDGWDPATGLGTPDFGKLLSLAMRR | |
| 4 | TRI039 | CDQVTTPQCLKDLYNIGDYKADPNGGSKVAFASFLEEYARYDDLAKFEE | Aspergillus |
| | mature | KLAPYAIGQNFSVTQYNGGLNDQNSASDSGEANLDLQYTVGVSSPTPVT | fumigatus |
| | Interpro | EFSTGGRGLLTPDLSQPDPNDNSNEPYLEFLQNVLKMDQDKLPQVTSIS | AF293 |
| | domain | YGEDEQTIPEKYARSVCNLYAQLGSRGVSVTFSSGDSGVGAACLTNDGT | |
| | IPR000209 | NRTHFPPQFPAACPWVTSVGGTTKTQPEEAVYFSSGGFSDLWERPSWQD | |
| | Peptidase | SAVKRYLKKLGPRYKGLYNPKGRAFPDVAAQAENYAVFDKGVLHQFDGT | |
| | S8/S53 dom | SCSAPAFSAIVALLNDARLRAHKPVMGFLNPWLYSKASKGFNDTVKGGS | |
| | | KGCDGRNRFGGTPNGSPVVPYASWNATDGWDPATGLGTPDFGKLLSLAM | |

The tripeptidyl peptidase (a) comprises the amino acid sequence SEQ ID No. 3, SEQ ID No. 4, or a functional fragment thereof; (b) comprises an amino acid having at least 70% identity to SEQ ID No. 3 or SEQ ID No. 4; (c) is encoded by a nucleotide sequence comprising the sequence SEQ ID No. 1 or SEQ ID No. 2; (d) is encoded by a nucleotide sequence which has at least about 70% identity to SEQ ID No. 1 or SEQ ID No. 2; (e) is encoded by a nucleotide sequence which hybridises to SEQ ID No. 1 or SEQ ID No. 2 under medium stringency conditions; or (f) is encoded by a nucleotide sequence which differs from SEQ ID No. 1 or SEQ ID No. 2 due to degeneracy of the genetic code. The tripeptidyl peptidase may be expressed as a polypeptide sequence which undergoes further post-transcriptional and/or post-translational modification.

In one embodiment, the tripeptidyl peptidase comprises the amino acid sequence SEQ ID No. 3, SEQ ID No. 4 or a functional fragment thereof.

In another embodiment, the tripeptidyl peptidase comprises an amino acid having at least 70% identity to SEQ ID No. 3, SEQ ID No. 4, or a functional fragment thereof.

In one embodiment, the tripeptidyl peptidase comprises the amino acid sequence SEQ ID SEQ ID No. 3 or a functional fragment thereof.

In another embodiment, the tripeptidyl peptidase comprises an amino acid having at least 70% identity to SEQ ID No. 3 or a functional fragment thereof.

In another embodiment, the tripeptidyl peptidase may be a “mature” tripeptidyl peptidase which has undergone post-transcriptional and/or post-translational modification (e.g. post-translational cleavage). Suitably such modification may lead to an activation of the enzyme.

Suitably, the tripeptidyl peptidase comprises the amino acid sequence SEQ ID No. 4 or a functional fragment thereof.

In another embodiment, the tripeptidyl peptidase comprises an amino acid having at least 70% identity to SEQ ID No. 4 or a functional fragment thereof.

As used herein, the term “functional fragment” is a portion of an amino acid sequence that retains its enzyme activity. Therefore, a functional fragment of a tripeptidyl peptidase is a portion of a tripeptidyl peptidase that is an exopeptidase capable of cleaving tripeptides from the N-terminus a peptide and/or proteins having one or more of lysine, arginine or glycine in the P1 position.

The “portion” is any portion that still has the activity as defined above; suitably a portion may be at least 50 amino acids in length, more suitably at least 100. In other embodiments the portion may be about 150 or about 200 amino acids in length.

In one embodiment, the functional fragment may be portion of a tripeptidyl peptidase following post transcriptional and/or post-translational modification (e.g. cleavage). Suitably, the functional fragment may comprise a sequence shown as SEQ ID No. 4.

In one embodiment, the tripeptidyl peptidase comprises one or more amino acid sequences selected from SEQ ID No. 3, or a functional fragment thereof.

In one embodiment, the tripeptidyl peptidase comprises an amino acid having at least 70% identity to SEQ ID No. 3 or a functional fragment thereof.

In one embodiment, the tripeptidyl peptidase comprises one or more amino acid sequence selected from SEQ ID No. 4, or a functional fragment thereof.

In one embodiment, the tripeptidyl peptidase comprises an amino acid having at least 70% identity to SEQ ID No. 4 or a functional fragment thereof.

In one embodiment, the tripeptidyl peptidase comprises an amino acid having at least 80% identity to SEQ ID No. 3, SEQ ID No. 4 or a functional fragment thereof.

In one embodiment, the tripeptidyl peptidase comprises an amino acid having at least 85% identity to SEQ ID No. 3, SEQ ID No. 4 or a functional fragment thereof.

In one embodiment, the tripeptidyl peptidase comprises an amino acid having at least 90% identity to SEQ ID No. 3, SEQ ID No. 4 or a functional fragment thereof.

In one embodiment, the tripeptidyl peptidase comprises an amino acid having at least 95% identity to SEQ ID No. 3, SEQ ID No. 4 or a functional fragment thereof.

In one embodiment, the tripeptidyl peptidase comprises an amino acid sequence selected from one more of the group consisting of: SEQ ID No. 3 or SEQ ID No. 4.

In one embodiment, the tripeptidyl peptidase is encoded by a nucleotide sequence SEQ ID No. 1, SEQ ID No. 2 or a nucleotide sequence having at least 70% identity thereto, suitably a sequence having at least 80% thereto or at least 90% thereto.

In a preferred embodiment, the tripeptidyl peptidase is encoded by a nucleotide sequence having at least 95% sequence identity to SEQ ID No. 1 or SEQ ID No. 2, more preferably at least 99% identity to SEQ ID No. 1 or SEQ ID No. 2.

In another embodiment, the tripeptidyl peptidase is encoded by a nucleotide sequence which hybridizes to SEQ ID No. 1 or SEQ ID No. 2 under medium stringency conditions. Suitably, a nucleotide sequence which hybridizes to SEQ ID No. 1 or SEQ ID No. 2 under high stringency conditions.

In a further embodiment, the tripeptidyl peptidase is encoded by a nucleotide sequence which differs from SEQ ID No. 1 or SEQ ID No. 2 due to degeneracy of the genetic code.

In one embodiment, the isolated polynucleotide comprises a nucleotide sequence shown as SEQ ID No. 1 or SEQ ID No. 2 may be a DNA, cDNA, synthetic DNA and/or RNA sequence.

Preferably, the sequence is a DNA sequence, more preferably a cDNA sequence coding for the tripeptidyl peptidase.

In another embodiment, an isolated nucleic acid is provided comprising:
(a) a nucleotide sequence as shown herein as SEQ ID No. 1 or SEQ ID No. 2;
(b) a nucleotide sequence which has at least about 70% identity to SEQ ID No. 1 or SEQ ID No. 2;
(c) a sequence that hybridises to SEQ ID No. 1 or SEQ ID No. 2 under medium stringency conditions; or
(c) a nucleotide sequence which differs from SEQ ID No. 1 or SEQ ID No. 2 due to degeneracy of the genetic code.

In one embodiment, the nucleotide sequence may be a nucleotide sequence having at least about 80% identity to SEQ ID No. 1 or SEQ ID No. 2; preferably at least about 90% identity to SEQ ID No. 1 or SEQ ID No. 2.

In a preferred embodiment, the nucleotide sequence may be a nucleotide sequence having at least bout 95% identity, suitably at least about 99% identity to SEQ ID No. 1 or SEQ ID No. 2.

In one embodiment, the isolated nucleic acid may comprise a nucleotide sequence that hybridises to SEQ ID No. 1 or SEQ ID No. 2 under high stringency conditions.

Suitably, the isolated nucleic acid may be comprised in a vector (for example, a plasmid).

In another embodiment, a Trichderma host cell is also provided comprising an isolated nucleic acid sequence or vector.

Preferably, the host cell may be a Trichderma reesei host cell.

In one preferred aspect, the amino acid and/or nucleotide sequence is in an isolated form. The term “isolated” means that the sequence is at least substantially free from at least one other component with which the sequence is naturally associated in nature and as found in nature. The amino acid and/or nucleotide sequence may be provided in a form that is substantially free of one or more contaminants with which the substance might otherwise be associated. Thus, for example it may be substantially free of one or more potentially contaminating polypeptides and/or nucleic acid molecules.

In one preferred aspect, the amino acid and/or nucleotide sequence is in a purified form. The term “purified” means that a given component is present at a high level. The component is desirably the predominant component present in a composition. Preferably, it is present at a level of at least about 90%, or at least about 95% or at least about 98%, said level being determined on a dry weight/dry weight basis with respect to the total composition under consideration.

### Enzymes

In one embodiment, the enzyme is a tripeptidyl peptidase comprising SEQ ID No. 3, a functional fragment thereof or a sequence having at least 70% identity to SEQ ID No. 3. Suitably the enzyme may have at least 80%, preferably at least 90% identity to SEQ ID No. 3.

In one embodiment, the enzyme is a tripeptidyl peptidase comprising SEQ ID No. 4, a functional fragment thereof or a sequence having at least 70% identity to SEQ ID No. 4. Suitably the enzyme may have at least 80%, preferably at least 90% identity to SEQ ID No. 4.

In one embodiment, the enzyme is a tripeptidyl peptidase encoded by a nucleotide sequence comprising the sequence shown as SEQ ID No. 1 or a sequence having at least 70% identity thereto; preferably at least 80% identity, and even more preferably at least 90% identity thereto.

In one embodiment, the enzyme is a tripeptidyl peptidase encoded by a nucleotide sequence comprising the sequence shown as SEQ ID No. 2 or a sequence having at least 70% identity thereto; preferably at least 80% identity, even more preferably at least 90% identity thereto.

### Nucleotide Sequence

In another embodiment, polynucleotides having nucleic acid sequences are provided encoding proteins having the specific properties as defined herein.

The term “nucleotide sequence” or “nucleic acid sequence”, as used herein, refers to an oligonucleotide sequence or polynucleotide sequence, and variant, homologues, fragments and derivatives thereof (such as portions thereof). The polynucleotides/oligonucleotides having nucleic acid sequences may be of genomic or synthetic or recombinant origin, which may be double-stranded or single-stranded whether representing the sense or anti-sense strand.

The term “nucleotide sequence” or “nucleic acid sequence” includes genomic DNA, cDNA, synthetic DNA, and RNA sequences. In a preferred aspect it means DNA sequences and more preferably cDNA sequences.

In a preferred embodiment, the polynucleotides do not include the native polynucleotides when in their natural environment and when it is linked to its naturally associated sequence(s) that is/are also in its/their natural environment. For ease of reference, this preferred embodiment will be referred to as the “non-native nucleotide sequence” or “non-native nucleic acid sequence”. In this regard, the term “native nucleotide sequence” or “native nucleic acid sequence” means an entire nucleic acid sequence encoding a nucleic acid molecule that is in its native environment and when operatively linked to an entire promoter with which it is naturally associated, which promoter is also in its native environment. However, the polypeptide having an amino acid sequence as described herein can be isolated and/or purified post expression of a polynucleotide in its native organism. Preferably, however, the amino acid sequence may be encoded by a nucleic acid sequence in its native organism but wherein the nucleic acid molecule is not under the control of the promoter with which it is naturally associated within that organism.

Typically, the polynucleotide molecules described herein having nucleotide sequences are prepared using recombinant DNA techniques (i.e. recombinant DNA). However, in an alternative embodiment of the invention, the polynucleotide molecules could be synthesised, in whole or in part, using chemical methods well known in the art (see Caruthers MH et al., (1980) Nuc Acids Res Symp Ser 215-23 and Horn T et al., (1980) Nuc Acids Res Symp Ser 225-232).

### Preparation of the Nucleotide Sequence

A polynucleotide having a nucleic acid sequence encoding either 1) a protein which has the specific properties as defined herein or 2) a protein which is suitable for modification may be identified and/or isolated and/or purified from any cell or organism producing said protein. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from the organism producing the enzyme. If the amino acid sequence of the enzyme is known, labelled oligonucleotide probes may be synthesised and used to identify enzyme-encoding clones from the genomic library prepared from the organism. Alternatively, a labelled oligonucleotide probe containing sequences homologous to another known enzyme gene could be used to identify enzyme-encoding clones. In the latter case, hybridisation and washing conditions of lower stringency are used.

Alternatively, enzyme-encoding clones could be identified by inserting fragments of genomic DNA into an expression vector, such as a plasmid, transforming enzyme-negative bacteria with the resulting genomic DNA library, and then plating the transformed bacteria onto agar plates containing a substrate for enzyme (i.e. maltose), thereby allowing clones expressing the enzyme to be identified.

In a yet further alternative, the nucleotide sequence encoding the enzyme may be prepared synthetically by established standard methods, e.g. the phosphoramidite method described by Beucage S. L. et al., (1981) Tetrahedron Letters 22:1859-1869, or the method described by Matthes et al., (1984) EMBO J. 3:801-805. In the phosphoramidite method, oligonucleotides are synthesised, e.g. in an automatic DNA synthesiser, purified, annealed, ligated and cloned in appropriate vectors.

The nucleic acid molecules may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al., (Science (1988) 239:487-491) the teaching of these documents being incorporated herein by reference.

### Amino Acid Sequences

The scope of the present invention also encompasses polypeptides having amino acid sequences of enzymes having the specific properties as defined herein.

As used herein, the term “protein” is synonymous with the term “polypeptide”, “oligopeptide” and/or the term “peptide”. In some instances, the term “polypeptide” (as defined by an enzymatic activity) is synonymous with the term “enzyme”.

The polypeptides having amino acid sequences may be prepared and/or isolated from a suitable source, or it may be made synthetically or it may be prepared by use of recombinant DNA techniques.

The protein encompassed in the present invention may be used in conjunction with other proteins, particularly enzymes. Thus the present invention also covers a combination of proteins wherein the combination comprises the protein/enzyme of the present invention and another protein/enzyme, which may be another protein/enzyme according to the present invention. This aspect is discussed in a later section.

Preferably the polypetide, when relating to and when encompassed by the per se scope described herein, is not a native enzyme. In this regard, the term “native enzyme” means an entire enzyme that is in its native environment and when it has been expressed by its native nucleotide sequence.

### Isolated

In one aspect, preferably the present polypeptide(s), nucleic acid molecule(s), and/or enzyme(s) are in an isolated form. The term “isolated” means that the polypeptide, enzyme, and/or nucleic acid molecule are at least substantially free from at least one other component with which materials are naturally associated and/or found in nature. The polypeptides, enzymes and/or nucleic acid molecules described herein may be provided in a form that is substantially free of one or more contaminants with which the substance might otherwise be associated. Thus, for example it may be substantially free of one or more potentially contaminating polypeptides and/or nucleic acid molecules.

### Purified

In one aspect, preferably the polypeptide(s), enzyme(s), and/or nucleic acid molecules are in a purified form. The term “purified” means that the given component is present at a high level. The component is desirably the predominant component present in a composition. Preferably, it is present at a level of at least about 80% said level being determined on a dry weight/dry weight basis with respect to the total composition under consideration. Suitably it may be present at a level of at least about 90%, or at least about 95, or at least about 98% said level being determined on a dry weight/dry weight basis with respect to the total composition under consideration.

### Sequence Identity or Sequence Homology

The present invention also encompasses the use of polypeptides and/or nucleic acid molecules having sequences having a degree of sequence identity or sequence homology with amino acid sequence(s) of a polypeptide having the specific properties defined herein or of any nucleotide sequence encoding such a polypeptide (hereinafter referred to as a “homologous sequence(s)”). Here, the term “homologue” means an entity having a certain homology with the subject amino acid sequences and the subject nucleotide sequences. Here, the term “homology” can be equated with “identity”.

The homologous amino acid sequence and/or nucleotide sequence should provide and/or encode a polypeptide which retains the functional activity and/or enhances the activity of the enzyme.

In the present context, a homologous sequence is taken to include an amino acid or a nucleotide sequence which may be at least 75, 85 or 90% identical, preferably at least 95 or 98% identical to the subject sequence. Typically, the homologues will comprise the same active sites etc. as the subject amino acid sequence for instance. Although homology can also be considered in terms of similarity (i.e. amino acid residues having similar chemical properties/functions), in the context of the present invention it is preferred to express homology in terms of sequence identity.

In one embodiment, a homologous sequence is taken to include an amino acid sequence or nucleotide sequence which has one or several additions, deletions and/or substitutions compared with the subject sequence.

In one embodiment the present invention relates to a protein whose amino acid sequence is represented herein or a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

Suitably, the degree of identity with regard to an amino acid sequence is determined over at least 20 contiguous amino acids, preferably over at least 30 contiguous amino acids, preferably over at least 40 contiguous amino acids, preferably over at least 50 contiguous amino acids, preferably over at least 60 contiguous amino acids, preferably over at least 100 contiguous amino acids, preferably over at least 200 contiguous amino acids.

In one embodiment the present invention relates to a nucleic acid sequence (or coding sequence) encoding a protein whose amino acid sequence is represented herein or encoding a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

In the present context, a homologous sequence is taken to include a nucleotide sequence which may be at least 75, 85 or 90% identical, preferably at least 95 or 98% identical to a nucleotide sequence encoding a polypeptide of the present invention (the subject sequence). Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % homology or % identity therefore firstly requires the production of an optimal alignment, taking into consideration gap penalties. A suitable computer program for carrying out such an alignment is Vector NTI® (Thermo Fisher Scientific, Waltham, Mass., USA). Examples of software that can perform sequence comparisons include, but are not limited to, the BLAST package (Ausubel, F. M. et. al., Short Protocols in Molecular Biology, 5th Ed. Current Protocols and John Wiley and Sons, Inc., N.Y., 2002), BLAST 2 (FEMS Microbiol Lett (1999) 174(2): 247-50; FEMS Microbiol Lett (1999) 177(1): 187-8), FASTA (Altschul et al., J. Mol. Biol. (1990) 215:403-410), and AlignX, for example. At least BLAST, BLAST 2 and FASTA are available for offline and online searching, such as for example in the GenomeQuest search tool (www.genomequest.com).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage homologies may be calculated using the multiple alignment feature in Vector NTI® (Thermo Fisher Scientific based on an algorithm, analogous to CLUSTAL (such as CLUSTALW (e.g., version 1.83; Thompson et al., Nucleic Acids Research, (1994) 22(22):4673-4680).

Once the software has produced an optimal alignment, it is possible to calculate % homology, preferably % sequence identity. The software typically does this as part of the sequence comparison and generates a numerical result.

Should Gap Penalties be used when determining sequence identity, then preferably the following parameters are used for pairwise alignment:

| FOR BLAST | |
|---|---|
| GAP OPEN | 9 |
| GAP EXTENSION | 2 |

| FOR CLUSTAL | DNA | PROTEIN |
|---|---|---|
| Weight Matrix | IUB | Gonnet 250 |
| GAP OPENING | 15 | 10 |
| GAP EXTEND | 6.66 | 0.1 |

In one embodiment, CLUSTAL may be used with the gap penalty and gap extension set as defined above.

Suitably, the degree of identity with regard to a nucleotide sequence is determined over at least 20 contiguous nucleotides, preferably over at least 30 contiguous nucleotides, preferably over at least 40 contiguous nucleotides, preferably over at least 50 contiguous nucleotides, preferably over at least 60 contiguous nucleotides, preferably over at least 100 contiguous nucleotides.

Suitably, the degree of identity with regard to a nucleotide sequence is determined over at least 100 contiguous nucleotides, preferably over at least 200 contiguous nucleotides, preferably over at least 300 contiguous nucleotides, preferably over at least 400 contiguous nucleotides, preferably over at least 500 contiguous nucleotides, preferably over at least 600 contiguous nucleotides, preferably over at least 700 contiguous nucleotides, preferably over at least 800 contiguous nucleotides.

Suitably, the degree of identity with regard to a nucleotide sequence may be determined over the whole sequence.

Suitably, the degree of identity with regard to a protein (amino acid) sequence is determined over at least 100 contiguous amino acids, preferably over at least 200 contiguous amino acids, preferably over at least 300 contiguous amino acids.

Suitably, the degree of identity with regard to an amino acid or protein sequence may be determined over the whole sequence taught herein.

In the present context, the term “query sequence” means a homologous sequence or a foreign sequence, which is aligned with a subject sequence in order to see if it falls within the scope of the present invention. Accordingly, such query sequence can for example be a prior art sequence or a third party sequence.

In one preferred embodiment, the sequences are aligned by a global alignment program and the sequence identity is calculated by identifying the number of exact matches identified by the program divided by the length of the subject sequence.

In one embodiment, the degree of sequence identity between a query sequence and a subject sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the subject sequence.

In yet a further preferred embodiment, the global alignment program is selected from the group consisting of CLUSTAL and BLAST (preferably BLAST) and the sequence identity is calculated by identifying the number of exact matches identified by the program divided by the length of the subject sequence.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by synthetic amino acids (e.g. unnatural amino acids) include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid#, 7-amino heptanoic acid*, L-methionine sulfone#*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid # and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the a-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The nucleic acid molecules described herein may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The present invention also encompasses the use of nucleotide sequences that are complementary to the sequences presented herein, or any derivative, fragment or derivative thereof. If the sequence is complementary to a fragment thereof then that sequence can be used as a probe to identify similar coding sequences in other organisms etc.

Polynucleotides which are not 100% homologous to the present sequences can be obtained in a number of ways. Other variants of the sequences described herein may be obtained for example by probing DNA libraries made from a range of individuals, for example individuals from different populations. In addition, other homologues may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

Polynucleotides (nucleotide sequences) may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. The primers may be designed to contain suitable restriction enzyme recognition sites so that the amplified DNA can be cloned into a suitable cloning vector.

### Hybridization

In one embodiment, the compositions and methods also encompass sequences that are complementary to the nucleic acid sequences described herein or sequences that are capable of hybridising either to the sequences described herein or to sequences that are complementary thereto.

The term “hybridisation” or “hybridization”, as used herein, shall include “the process by which a strand of nucleic acid joins with a complementary strand through base pairing” as well as the process of amplification as carried out in polymerase chain reaction (PCR) technologies.

In one embodiment, the use of nucleotide sequences that are capable of hybridising to the sequences that are complementary to the sequences presented herein, or any derivative, fragment or derivative thereof as also provided

The term “variant” also encompasses sequences that are complementary to sequences that are capable of hybridising to the nucleotide sequences presented herein.

Hybridization and washing conditions are well known and exemplified in Sambrook, J. and Russell, D., T. Molecular Cloning: A Laboratory Manual, Third Edition, Cold Spring Harbor Laboratory Press, Cold Spring Harbor (2001). The conditions of temperature and ionic strength determine the “stringency” of the hybridization. Stringency conditions can be adjusted to screen for moderately similar molecules, such as homologous sequences from distantly related organisms, to highly similar molecules, such as genes that duplicate functional enzymes from closely related organisms. Post-hybridization washes typically determine stringency conditions. One set of preferred conditions uses a series of washes starting with 6X× SSC, 0.5% SDS at room temperature for 15 min, then repeated with 2× SSC, 0.5% SDS at 45° C. for 30 min, and then repeated twice with 0.2× SSC, 0.5% SDS at 50° C. for 30 min. A more preferred set of conditions uses higher temperatures in which the washes are identical to those above except for the temperature of the final two 30 min washes in 0.2× SSC, 0.5% SDS was increased to 60° C. Another preferred set of high stringent hybridization conditions is 0.1× SSC, 0.1% SDS, 65° C. and washed with 2× SSC, 0.1% SDS followed by a final wash of 0.1× SSC, 0.1% SDS, 65° C.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (Sambrook, J. and Russell, D., T., supra). For hybridizations with shorter nucleic acids, i.e., oligonucleotides, the position of mismatches becomes more important, and the length of the oligonucleotide determines its specificity. In one aspect, the length for a hybridizable nucleic acid is at least about 10 nucleotides. Preferably, a minimum length for a hybridizable nucleic acid is at least about 15 nucleotides in length, more preferably at least about 20 nucleotides in length, even more preferably at least 30 nucleotides in length, even more preferably at least 300 nucleotides in length, and most preferably at least 800 nucleotides in length. Furthermore, the skilled artisan will recognize that the temperature and wash solution salt concentration may be adjusted as necessary according to factors such as length of the probe.

Preferably hybridisation is analysed over the whole of the sequences taught herein.

### Molecular Evolution

As a non-limiting example, it is possible to produce numerous site directed or random mutations into a nucleotide sequence, either in vivo or in vitro, and to subsequently screen for improved functionality of the encoded polypeptide by various means.

In addition, mutations or natural variants of a polynucleotide sequence can be recombined with either the wildtype or other mutations or natural variants to produce new variants. Such new variants can also be screened for improved functionality of the encoded polypeptide. The production of new preferred variants can be achieved by various methods well established in the art, for example the Error Threshold Mutagenesis (WO 92/18645), oligonucleotide mediated random mutagenesis (U.S. Pat. No. 5,723, 323), DNA shuffling (U.S. Pat. No. 5,605,793), and exo-mediated gene assembly WO00/58517. The application of these and similar random directed molecular evolution methods allows the identification and selection of variants of the enzymes described herein which have preferred characteristics without any prior knowledge of protein structure or function, and allows the production of non-predictable but beneficial mutations or variants. There are numerous examples of the application of molecular evolution in the art for the optimisation or alteration of enzyme activity, such examples include, but are not limited to one or more of the following: optimised expression and/or activity in a host cell or in vitro, increased enzymatic activity, altered substrate and/or product specificity, increased or decreased enzymatic or structural stability, altered enzymatic activity/specificity in preferred environmental conditions, e.g. temperature, pH, substrate.

### Site-Directed Mutagenesis

Once a protein-encoding nucleotide sequence has been isolated, or a putative protein-encoding nucleotide sequence has been identified, it may be desirable to mutate the sequence in order to prepare a protein.

Mutations may be introduced using synthetic oligonucleotides. These oligonucleotides contain nucleotide sequences flanking the desired mutation sites.

A suitable method is disclosed in Morinaga et al., (Biotechnology (1984) 2:646-649). Another method of introducing mutations into enzyme-encoding nucleotide sequences is described in Nelson and Long (Analytical Biochemistry (1989), 180:147-151).

### Recombinant

In one aspect, the sequence is a recombinant sequence—i.e. a sequence that hasbeen prepared using recombinant DNA techniques.

These recombinant DNA techniques are within the capabilities of a person of ordinary skill in the art. Such techniques are explained in the literature, for example, Sambrook, J. and Russell, D., T. Molecular Cloning: A Laboratory Manual, Third Edition, Cold Spring Harbor Laboratory Press, Cold Spring Harbor (2001).

### Synthetic

In one aspect, the sequence is a synthetic sequence—i.e. a sequence that has been prepared by in vitro chemical or enzymatic synthesis. It includes, but is not limited to, sequences made with optimal codon usage for host organisms—such as the methylotrophic yeasts Pichia and Hansenula.

Proteins and/or peptides may also be of a synthetic origin.

### Expression of Enzymes

In one embodiment, a method for the expression of a tripeptidyl peptidase is also provided, which tripeptidyl peptidase is an exoprotease and is capable of cleaving tripeptides from the N-terminus a peptide and/or proteins having one or more of lysine, arginine or glycine in the P1 position and wherein said method comprises:
(a) transforming a Trichderma host cell with a nucleic acid or vector comprising the nucleotide sequence SEQ ID No. 1 or SEQ ID No. 2; or a nucleotide sequence which has at least about 70% identity to SEQ ID No. 1 or SEQ ID No. 2; or a nucleotide sequence which hybridises to SEQ ID No. 1 or SEQ ID No. 2 under medium stringency conditions; or a nucleotide sequence which differs from SEQ ID No. 1 or SEQ ID No. 2 due to degeneracy of the genetic code;
(b) expressing the nucleic acid sequence or vector of step (a); and
(c) obtaining the tripeptidyl peptidase or a fermentate comprising said tripeptidyl peptidase.

Suitably the method may further comprise isolating and/or purifying and/or packaging the tripeptidyl peptidase.

The nucleic acid may be any nucleic acid encoding a tripeptidyl peptidase having the activity detailed herein. Suitably, the nucleic acid molecule may be any one of the nucleic acids detailed herein. Suitably, the nucleic acid molecule may be an isolated nucleic acid as described herein.

The nucleic acid molecule may be incorporated into a recombinant replicable vector. The vector may be used to replicate and express the nucleotide sequence, in protein/enzyme form, in and/or from a compatible host cell.

Expression may be controlled using control sequences, such as regulatory sequences.

The protein produced by a host recombinant cell by expression of the nucleotide sequence may be secreted or may be contained intracellularly depending on the sequence and/or the vector used. The coding sequences may be designed with signal sequences which direct secretion of the substance coding sequences through a particular prokaryotic or eukaryotic cell membrane.

The term “expression vector” means a construct capable of in vivo or in vitro expression.

In one embodiment, the tripeptidyl peptidase and/or endoprotease may be encoded by a vector. In other words the vector may comprise a nucleotide sequence encoding the tripeptidyl peptidase.

Preferably, the expression vector is incorporated into the genome of a suitable host organism. The term “incorporated” preferably covers stable incorporation into the genome.

The nucleic acid molecules may be present in a vector in which the nucleotide sequence is operably linked to regulatory sequences capable of providing for the expression of the nucleotide sequence by a suitable host organism.

The vectors for use in the present invention may be transformed into a suitable host cell as described below to provide for expression of a polypeptide of the present invention.

The choice of vector e.g. a plasmid, cosmid, or phage vector will often depend on the host cell into which it is to be introduced.

The vectors may contain one or more selectable marker genes—such as a gene, which confers antibiotic resistance e.g. ampicillin, kanamycin, chloramphenicol or tetracyclin resistance. Alternatively, the selection may be accomplished by co-transformation (as described in WO91/17243).

Vectors may be used in vitro, for example for the production of RNA or used to transfect, transform, transduce or infect a host cell.

Thus, in a further embodiment, a method of making nucleic acid molecules is also provided by introducing a polynucleotide as described herein into a replicable vector, introducing the vector into a compatible host cell, and growing the host cell under conditions which bring about replication of the vector.

The vector may further comprise genetic elements enabling the vector to replicate in the host cell in question. Examples of such sequences are the origins of replication of plasmids pUC19, pACYC177, pUB110, pE194, pAMB1 and pIJ702.

The nucleotide sequence and/or vector encoding the tripeptidyl peptidase and/or the endoprotease may be codon optimised for expression in a particular host organism.

The nucleotide sequence and/or vector encoding the tripeptidyl peptidase and/or the endoprotease may be codon optimised for expression in a prokaryotic or eukaryotic cell. Suitably, the nucleotide sequence and/or vector encoding the tripeptidyl peptidase and/or the endoprotease may be codon optimised for expression in a fungal host organism (e.g. Trichderma, preferably Trichderma reesei).

Codon optimisation refers to a process of modifying a nucleic acid sequence for enhanced expression in a host cell of interest by replacing at least one codon (e.g. at least about more than 1, 2, 3, 4, 5, 10, 15, 20, 25, 50, 60, 70, 80 or 100 codons) of the native sequence with codons that are more frequently used in the genes of the host cell, whilst maintaining the native amino acid sequence. Various species exhibit particular bias for certain codons of a particular amino acid. Codon bias (differences in codon usage between organisms) often correlates with the efficiency of translation of messenger RNA (mRNA), which is in tum believed to be dependent on, amongst other things, the properties of the codons being translated and the availability of particular transfer RNA (tRNA) molecules. The predominance of selected tRNAs in a cell is generally a reflection of the codons used most frequently in peptide synthesis.

Accordingly, genes can be tailored for optimal gene expression in a given organism based on codon optimisation. A nucleotide sequence and/vector that has undergone this tailoring can be referred to therefore as a “codon optimised” nucleotide sequence and/or vector.

Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimising a particular sequence for expression in a particular host cell are also available, such as Gene Forge™ (Aptagen; Jacobus, PA, USA). In some embodiments, one or more codons (e.g. 1, 2, 3, 4, 5, 10, 15, 20, 25, 50, or more, or all codons) in a sequence encoding a tripeptidyl peptidase and/or endoprotease correspond to the most frequently used codon for a particular amino acid.

In one embodiment the nucleotide sequence encoding the tripeptidyl peptidase may be a nucleotide sequence which has been codon optimized for expression in Trichderma reesei.

In one embodiment the codon optimized sequence may comprise a nucleotide sequence shown as SEQ ID No. 2 or a nucleotide sequence having at least 70% identity thereto, suitably a sequence having at least 80% thereto or at least 90% thereto.

Preferably the codon optimized sequence may comprise a nucleotide sequence having at least 95% sequence identity to SEQ ID No. 2.

In one embodiment the tripeptidyl peptidase may be encoded by a nucleotide sequence which hybridizes to SEQ ID No. 2 under medium stringency conditions. Suitably, a nucleotide sequence which hybridizes to SEQ ID No. 2 under high stringency conditions.

In a further embodiment, the tripeptidyl peptidase may be encoded by a nucleotide sequence which differs from SEQ ID No. 2 due to degeneracy of the genetic code.

### Regulatory Sequences

In some applications, the polynucleotide molecule is operably linked to a regulatory sequence which is capable of providing for the expression of the nucleotide sequence, such as by the chosen host cell. In another embodiment, a vector is provided comprising a nucleic acid molecule as described herein operably linked to such a regulatory sequence, i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences.

The term “regulatory sequences” includes promoters and enhancers and other expression regulation signals.

The term “promoter” is used in the normal sense of the art, e.g. an RNA polymerase binding site.

Enhanced expression of the nucleotide sequence encoding at least one of enzyme(s) described herein may also be achieved by the selection of heterologous regulatory regions, e.g. promoter, secretion leader and terminator regions.

Preferably, the nucleotide sequence according to the present invention is operably linked to at least a promoter.

Other promoters may even be used to direct expression of the polypeptide(s) described herein.

Examples of suitable promoters for directing the transcription of the nucleotide sequence in a bacterial, fungal or yeast host are well known in the art.

The promoter can additionally include features to ensure or to increase expression in a suitable host. For example, the features can be conserved regions such as a Pribnow Box or a TATA box.

### Constructs

As used herein, the term “construct”—which is synonymous with terms such as “conjugate”, “cassette” and “hybrid”—includes a polynucleotide having a nucleotide sequence directly or indirectly attached to a promoter.

An example of an indirect attachment is the provision of a suitable spacer group such as an intron sequence, such as the Sh1-intron or the ADH intron, intermediate the promoter and the nucleotide sequence of the present invention. The same is true for the term “fused” which includes direct or indirect attachment. In some cases, the terms do not cover the natural combination of the nucleotide sequence coding for the protein ordinarily associated with the wild type gene promoter and when they are both in their natural environment.

The construct may even contain or express a marker, which allows for the selection of the genetic construct.

For some applications, preferably the construct comprises at least one of the polynucleotides described herein operably linked to a promoter.

### Host Cells

The term “host cell”—includes any cell that comprises either the nucleotide sequence or an expression vector as described above and which is used in the recombinant production of a protein having the specific properties as defined herein.

Thus, a further embodiment provides host cells transformed or transfected with a nucleotide sequence that expresses at least one of the present proteins. The cells will be chosen to be compatible with the said vector and may for example be prokaryotic (for example bacterial), fungal, yeast or plant cells.

Examples of suitable bacterial host organisms are gram positive or gram negative bacterial species.

Depending on the nature of the nucleotide sequence encoding the present polypeptide, and/or the desirability for further processing of the expressed protein, eukaryotic hosts such as yeasts or other fungi may be preferred. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

The use of suitable host cells—such as yeast, fungal and plant host cells—may provide for post-translational modifications (e.g. myristoylation, glycosylation, truncation, lipidation and tyrosine, serine or threonine phosphorylation) as may be needed to confer optimal biological activity on recombinant expression products of the present invention.

The host cell may be a protease deficient or protease minus strain. This may for example be the protease deficient strain Aspergillus oryzae JaL 125 having the alkaline protease gene named “alp” deleted. This strain is described in WO97/35956.

### Organism

As used herein, the term “organism” includes any organism that could comprise the nucleotide sequence coding for the polypeptide according to the present invention and/or products obtained therefrom, and/or wherein a promoter can allow expression of the nucleotide sequence according to the present invention when present in the organism.

Suitable organisms may include a prokaryote, fungus, yeast or a plant.

As used herein, the term “transgenic organism” includes any organism that comprises the nucleotide sequence coding for the polypeptide according to the present invention and/or the products obtained therefrom, and/or wherein a promoter can allow expression of a polynucleotide within the organism. Preferably the nucleotide sequence is incorporated in the genome of the organism.

The term “transgenic organism” does not cover native nucleotide coding sequences in their natural environment when they are under the control of their native promoter which is also in its natural environment.

Therefore, the transgenic organism includes an organism comprising any one of, or combinations of, the nucleotide sequence coding for the polypeptide(s) described herein, constructs, vectors, plasmids, cells, tissues, and/or the products thereof.

For example the transgenic organism may also comprise the polynucleotides coding for the polypeptide described herein under the control of a heterologous promoter.

### Transformation of Host Cells/Organism

As indicated earlier, the host organism is a Trichderma, preferably Trichderma reesei.

Fungal cells may be transformed using various methods known in the art—such as a process involving protoplast formation and transformation of the protoplasts followed by regeneration of the cell wall in a manner known.

General teachings on the transformation of fungi are presented in following sections.

### Transformed Fungus

A host organism may be a fungus—such as a Trichderma and the like.

Suitably the host organism is a Trichderma host organism, for example, a Trichderma reesei host organism.

### Culturing and Production

Host cells transformed with the nucleotide sequence of the present invention may be cultured under conditions conducive to the production of the encoded polypeptide and which facilitate recovery of the polypeptide from the cells and/or culture medium.

The medium used to cultivate the cells may be any conventional medium suitable for growing the host cell in questions and obtaining expression of the polypeptide.

The protein produced by a recombinant cell may be displayed on the surface of the cell.

The protein may be secreted from the host cells and may conveniently be recovered from the culture medium using well-known procedures.

### Secretion

Often, it is desirable for the protein to be secreted from the expression host into the culture medium from where the protein may be more easily recovered. The secretion leader sequence may be selected on the basis of the desired expression host. Hybrid signal sequences may also be used with the context of the present compositions and methods.

Typical examples of heterologous secretion leader sequences are those originating from the fungal amyloglucosidase (AG) gene (glaA—both 18 and 24 amino acid versions e.g. from Aspergillus), the a-factor gene (yeasts e.g. Saccharomyces, Kluyveromyces and Hansenula) or the a-amylase gene (Bacillus).

By way of example, the secretion of heterologous proteins in E. coli is reviewed in Methods Enzymol (1990) 182:132-43.

### Post-transcription and Post-translational Modifications

Suitably the tripeptidyl peptidase and/or the endoprotease for may be encoded by any one of the nucleotide sequences taught herein.

Depending upon the host cell used post-transcriptional and/or post-translational modifications may be made. It is envisaged that the enzymes (e.g. the tripeptidyl peptidase and/or the endoprotease) for use in the present methods and/or uses encompasses enzymes (e.g. the tripeptidyl peptidase and/or the endoprotease) which have undergone post-transcriptional and/or post-translational modification.

One non-limiting example of a post-transcriptional and/or post-translational modifications is “clipping” or “cleavage” of a polypeptide (e.g. of the tripeptidyl peptidase and/or the endoprotease).

In some embodiments, the polypeptide (e.g. the tripeptidyl peptidase of the present invention e.g. tripeptidyl peptidase and/or the endoprotease) may be clipped or cleaved. This may result in the conversion of the tripeptidyl peptidase and/or the endoprotease from an inactive or substantially inactive state to an active state (i.e. capable of performing the activity described herein).

The tripeptidyl peptidase may be a pro-peptide which undergoes further post-translational modification to a mature peptide, i.e. a polypeptide which has the tripeptidyl peptidase activity.

By way of example only, SEQ ID No. 3 is the same as SEQ ID No. 4 except that SEQ ID No. 3 has undergone post-translational and/or post-transcriptional modification to remove some amino acids, more specifically some amino acids from the N-terminus. Therefore the polypeptide shown herein as SEQ ID No. 3 could be considered in some circumstances (i.e. in some host cells) as a pro-peptide—which is further processed to a mature peptide (SEQ ID No. 4) by post-translational and/or post-transcriptional modification. The precise modifications, e.g. cleavage site(s), in respect of the post-translational and/or post-transcriptional modification may vary slightly depending on host species. In some host species there may be no post translational and/or post-transcriptional modification, hence the pro-peptide would then be equivalent to the mature peptide (i.e. a polypeptide which has the tripeptidyl peptidase activity of the present invention). Without wishing to be bound by theory, the cleavage site(s) may be shifted by a few residues (e.g. 1, 2 or 3 residues) in either direction compared with the cleavage site shown by reference to SEQ ID No. 4 compared with SEQ ID No. 3.

Other examples of post-transcriptional and/or post-translational modifications include but are not limited to myristoylation, glycosylation, truncation, lipidation and tyrosine, serine or threonine phosphorylation. The skilled person will appreciate that the type of post-transcriptional and/or post-translational modifications that may occur to a protein (e.g. the tripeptidyl peptidase and/or the endoprotease) may depend on the host organism in which the protein (e.g. the tripeptidyl peptidase and/or the endoprotease) is expressed.

### Detection

A variety of protocols for detecting and measuring the expression of the amino acid sequence are known in the art. Examples include enzyme-linked immunosorbent assay (ELISA), radioimmunoassay (RIA) and fluorescent activated cell sorting (FACS).

A wide variety of labels and conjugation techniques are known by those skilled in the art and can be used in various nucleic and amino acid assays.

A number of companies such as Pharmacia Biotech (Piscataway, N.J.), Promega (Madison, Wis.), and US Biochemical Corp (Cleveland, Ohio) supply commercial kits and protocols for these procedures.

Suitable reporter molecules or labels include those radionuclides, enzymes, fluorescent, chemiluminescent, or chromogenic agents as well as substrates, cofactors, inhibitors, magnetic particles and the like. Patents teaching the use of such labels include, but are not limited to U.S. Pat. Nos. 3,817,837; 3,850,752; 3,939,350; 3,996,345; 4,277,437; 4,275,149 and 4,366,241.

Also, recombinant immunoglobulins may be produced as shown in U.S. Pat. 4,816,567.

### Fusion Proteins

The amino acid sequence may be produced as a fusion protein, for example to aid in extraction and purification. Examples of fusion protein partners include glutathione-S-transferase (GST), 6×His, GAL4 (DNA binding and/or transcriptional activation domains) and (β-galactosidase). It may also be convenient to include a proteolytic cleavage site between the fusion protein partner and the protein sequence of interest to allow removal of fusion protein sequences.

Preferably, the fusion protein will not hinder the activity of the protein sequence.

Gene fusion expression systems in E. coli have been reviewed in Curr Opin Biotechnol (1995) 6(5):501-6.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognised by a commercially available antibody.

### General Recombinant DNA Methodology Techniques

Unless otherwise indicated, conventional techniques of chemistry, molecular biology, microbiology, recombinant DNA and immunology, which are within the capabilities of a person of ordinary skill in the art. Such techniques are explained in the literature. See, for example, Sambrook, J. and Russell, D., T. Molecular Cloning: A Laboratory Manual, Third Edition, Cold Spring Harbor Laboratory Press, Cold Spring Harbor (2001); Ausubel, F. M. et. al., Short Protocols in Molecular Biology, 5th Ed. Current Protocols and John Wiley and Sons, Inc., N.Y., (2002); Silhavy, T. J., Bennan, M. L. and Enquist, L. W., Experiments with Gene Fusions, Cold Spring Harbor Laboratory, Cold Press Spring Harbor, NY (1984); B. Roe, J. Crabtree, and A. Kahn, (1996), DNA Isolation and Sequencing: Essential Techniques, John Wiley and Sons Inc., N.Y.; M. J. Gait (Editor), (1984), Oligonucleotide Synthesis: A Practical Approach, Irl Press; and D. M. J. Lilley and J. E. Dahlberg (Editors), (1992), DNA Structure Part A: Synthesis and Physical Analysis of DNA Volume 211 (Methods in Enzymology), Academic Press, San Diego, Calif.

### Dosages

The tripeptidyl peptidase and/or the endoprotease for use in the methods and/or uses described herein may be dosed in any suitable amount.

In one embodiment, the tripeptidyl peptidase may be dosed in an amount of about 5 mg to 3 g of enzyme per kg of protein substrate and/or food and/or feed additive composition.

In the preparation of a hydrolysate suitably the enzyme tripeptidyl peptidase may be dosed in an amount of 5 mg to 3 g of enzyme per kg of protein substrate.

In one embodiment, suitably the enzyme tripeptidyl peptidase may be dosed in an amount of 25 mg to 1000 mg of enzyme per kg of protein substrate.

In another embodiment, the tripeptidyl peptidase may be dosed in an amount of about 1 mg to about 1 kg of enzyme per kg of food and/or feed and/or feedstuff and/or premix. Suitably the tripeptidyl peptidase may be dosed at about 1 mg to about 250 g per kg of food and/or feed and/or feedstuff and/or premix. Preferably, at about 1 mg to about 100 g (more preferably at about 1 mg to about 1 g) per kg of food and/or feed and/or feedstuff and/or premix.

The endoprotease may be dosed in an amount of about 50 to about 3000 mg of enzyme per kg of protein substrate, e.g. 0.05 to 3 g of enzyme per metric ton (MT) of protein substrate.

Suitably, the endoprotease may be dosed in an amount of less than about 4.0 g of enzyme per MT of protein substrate.

In another embodiment, the endoprotease may be dosed at between about 0.5 g and about 5.0 g of enzyme per MT of protein substrate. Suitably, the endoprotease may be dosed at between about 0.5 g and about 3.0 g of enzyme per MT of protein substrate. More suitably, the endoprotease may be dosed at about 1.0 g to about 2.0 g of enzyme per MT of protein substrate.

In one embodiment, the aminopeptidase may be dosed in an amount of between about 0.5 mg to about 2 g of enzyme per kg of protein substrate and/or food and/or feed additive composition. Suitably, the aminopeptidase may be dosed in an amount of between about 1 mg to about 2 g of enzyme per kg of protein substrate and/or food and/or feed additive composition; more suitably in an amount of between about 5 mg to about 1.5 g of enzyme per kg of protein substrate and/or food and/or feed additive composition.

In the preparation of a hydrolysate, the aminopeptidase may be dosed in an amount of between about 0.5 mg to about 2 g of enzyme per kg of protein substrate. Suitably, the aminopeptidase may be dosed in an amount of between about 1 mg to about 2 g of enzyme per kg of protein substrate; more suitably in an amount of between about 5 mg to about 1.5 g of enzyme per kg of protein substrate.

In one embodiment, the aminopeptidase may be dosed in an amount of between about 5 mg to about 500 mg of enzyme per kg of protein substrate. Suitably the aminopeptidase may be dosed in an amount of between about 50 mg to about 500 mg of enzyme per kg of protein substrate. Suitably the aminopeptidase may be dosed in an amount of between about 100 mg to about 450 mg of enzyme per kg of protein substrate.

### Subject

The term “subject” may be used to refer to an “animal” or a “human”.

Suitably, the subject may be a “sensitive individual” predisposed to having an immune reaction to an untreated hydrolysate comprising one or more particular proteins or portions thereof. For example, the subject may be a sensitive individual having: a gluten (e.g. gliadin) allergy, a milk protein allergy and/or a soy protein allergy.

The term “animal”, as used herein, means an animal that is to be or has been administered with a feed additive composition according to the present invention or a feedstuff comprising said feed additive composition according to the present invention.

Preferably, the animal is a mammal, a ruminant animal, monogastric animal, fish or crustacean including for example livestock or a domesticated animal (e.g. a pet).

In one embodiment the “animal” is livestock.

The term “livestock”, as used herein refers to any farmed animal. Preferably, livestock is one or more of cows or bulls (including calves), pigs (including piglets, swine, growing pigs, sows), poultry (including broilers, chickens, egg layers and turkeys), birds, fish (including freshwater fish, such as salmon, cod, trout and carp, e.g. koi carp, and marine fish, such as sea bass), crustaceans (such as shrimps, mussels and scallops), horses (including race horses), sheep (including lambs).

In another embodiment, the “animal” is a domesticated animal or pet or an animal maintained in a zoological environment.

The term “domesticated animal or pet or animal maintained in a zoological environment” as used herein refers to any relevant animal including canines (e.g. dogs), felines (e.g. cats), rodents (e.g. guinea pigs, rats, mice), birds, fish (including freshwater fish and marine fish), and horses.

In one embodiment, the animal is a monogastric animal. In a preferred embodiment the monogastric animal may be poultry or pig (or a combination thereof).

In another embodiment, the animal is a ruminant animal.

The term animal is not intended to refer to a human being.

### Formulations

A composition and/or food additive composition and/or feed additive composition may comprise a tripeptidyl peptidase and/or a hydrolysate produced as described herein.

In another embodiment, there is provided a composition and/or food additive and/or feed additive composition comprising a hydrolysate of the invention. Suitably, such a food and/or feed additive composition may further comprise a tripeptidyl peptidase (optionally in combination with an endoprotease).

The tripeptidyl peptidase for use in the methods and/or uses and/or the composition and/or food additive and/or feed additive composition may be formulated in any appropriate manner known in the art.

Typical liquid formulations of food grade enzymes may include the following components (% is in w/w): enzyme of interest 0.2%-30%; preferably 2%-20%.

The stability of the enzyme formulation might also be increased by using salts like NaCl, KCl, CaCl2, Na2SO4 or other food grade salts in concentrations from about 0.1% to about 20% (suitably from about 0.1% to about 5%). Without wishing to be bound by theory, it is believed that the high salt concentrations might again be a way of achieving microbial stability either alone or in combination with further ingredients. The mechanism of action may be due to lower water activity or a specific action between a certain enzyme and a salt. Therefore in some embodiments the tripeptidyl peptidase may be admixed with at least one salt.

Suitably the preservative may be sodium benzoate and/or potassium sorbate. These preservatives can be typically used in a combined concentration of about 0.1-1%, suitably about 0.2-0.5%. Sodium benzoate is most efficient at pH <5.5 and sodium sorbate at pH <6.

Suitably the sugar is sorbitol.

Suitably the salt is sodium sulphate.

In one embodiment, the one or more ingredients (e.g. used for the formulation of the composition and/or food additive composition and/or feed additive composition) may be selected from the group consisting of: polyols, such as glycerol and/or sorbitol; sugars, such as glucose, fructose, sucrose, maltose, lactose and trehalose; salts, such as NaCl, KCl, CaCl2, Na2SO4 or other food grade salts; a preservative, e.g. sodium benzoate and/or potassium sorbate or any combination thereof.

In a preferred embodiment, a composition is provided (e.g. a feed additive composition) or the use thereof and methods of making the same comprising an enzyme of the present invention formulated with a compound selected from one or more of the group consisting of: Na2SO4, NaH2PO4, Na2HPO4, Na3PO4, (NH4)H2PO4, K2HPO4, KH2PO4, K2SO4, KHSO4, ZnSO4, MgSO4, CuSO4, Mg(NO3)2, (NH4)2SO4, sodium borate, magnesium acetate, sodium citrate or any combination thereof.

Suitably, the one or more ingredients (e.g. used for the formulation of the composition and/or food additive composition and/or feed additive composition) may be selected from the group consisting of: a wheat carrier, sorbitol and sodium sulphate.

Suitably, the tripeptidyl peptidase and/or the composition and/or food additive and/or feed additive composition may be admixed with a wheat carrier.

Suitably, the tripeptidyl peptidase and/or the composition and/or food additive and/or feed additive composition may be admixed with sorbitol.

Suitably, the tripeptidyl peptidase and/or the composition and/or food additive and/or feed additive composition may be admixed with sodium sulphate.

In a preferred embodiment, the composition and/or food additive and/or feed additive composition may further comprise any endoprotease detailed herein.

### Forms

The feed additive composition and other components and/or the feedstuff comprising same may be used in any suitable form.

The feed additive composition may be used in the form of solid or liquid preparations or alternatives thereof. Examples of solid preparations include powders, pastes, boluses, capsules, pellets, tablets, dusts, and granules which may be wettable, spray-dried or freeze-dried. Examples of liquid preparations include, but are not limited to, aqueous, organic or aqueous-organic solutions, suspensions and emulsions.

In some applications, feed additive composition of the present invention may be mixed with feed or administered in the drinking water.

Suitable examples of forms include one or more of: powders, pastes, boluses, pellets, tablets, pills, granules, capsules, ovules, solutions or suspensions, which may contain flavouring or colouring agents, for immediate-, delayed-, modified-, sustained-, pulsed- or controlled-release applications.

By way of example, if the composition is used in a solid, e.g. pelleted form, it may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Preferred excipients for the forms include lactose, starch, a cellulose, milk sugar or high molecular weight polyethylene glycols.

For aqueous suspensions and/or elixirs, the composition may be combined with various sweetening or flavouring agents, colouring matter or dyes, with emulsifying and/or suspending agents and with diluents such as water, propylene glycol and glycerin, and combinations thereof.

Combination with Other Components

The tripeptidyl peptidase and endoprotease and/or the composition and/or food additive composition and/or feed additive composition and/or hydrolysate may be used in combination with other components.

In another preferred embodiment, the tripeptidyl peptidase and endoprotease and/or food additive composition and/or feed additive composition and/or hydrolysate may be used in combination with other components which are suitable for animal or human consumption and are capable of providing a medical or physiological benefit to the consumer.

In one embodiment the “another component” may be one or more enzymes.

Suitable additional enzymes may be one or more of the enzymes selected from the group consisting of: endoglucanases (E.C. 3.2.1.4); cellobiohydrolases (E.C. 3.2.1.91), β-glucosidases (E.C. 3.2.1.21), cellulases (E.C. 3.2.1.74), lichenases (E.C. 3.2.1.73), lipases (E.C. 3.1.1.3), lipid acyltransferases (generally classified as E.C. 2.3.1.x), phospholipases (E.C. 3.1.1.4, E.C. 3.1.1.32 or E.C. 3.1.1.5), phytases (e.g. 6-phytase (E.C. 3.1.3.26) or a 3-phytase (E.C. 3.1.3.8), alpha-amylases (E.C. 3.2.1.1), xylanases (E.C. 3.2.1.8, E.C. 3.2.1.32, E.C. 3.2.1.37, E.C. 3.1.1.72, or E.C. 3.1.1.73), glucoamylases (E.C. 3.2.1.3), proteases (for example subtilisin (E.C. 3.4.21.62) or a bacillolysin (E.C. 3.4.24.28) or an alkaline serine protease (E.C. 3.4.21.x) or a keratinase (E.C. 3.4.x.x)) and/or mannanases (e.g. a β-mannanase (E.C. 3.2.1.78)).

Suitably, the other component may be a phytase (for example a 6-phytase (E.C. 3.1.3.26) or a 3-phytase (E.C. 3.1.3.8)).

In one embodiment (particularly for feed applications) the other component may be one or more of the enzymes selected from the group consisting of xylanases (E.C. 3.2.1.8, E.C. 3.2.1.32, E.C. 3.2.1.37, E.C. 3.1.1.72, or E.C. 3.1.1.73), an amylase (including α-amylases (E.C. 3.2.1.1), G4-forming amylases (E.C. 3.2.1.60), β-amylases (E.C. 3.2.1.2) and γ-amylases (E.C. 3.2.1.3); and/or a protease (for example subtilisin (E.C. 3.4.21.62) or a bacillolysin (E.C. 3.4.24.28) or an alkaline serine protease (E.C. 3.4.21.x) or a keratinase (E.C. 3.4.x.x)).

In one embodiment (particularly for feed applications), the other component may be acombination of an amylase (for example a-amylases (E.C. 3.2.1.1)) and a protease (for example subtilisin (E.C. 3.4.21.62)).

In one embodiment (particularly for feed applications) the other component may be a β-glucanase, such as an endo-1,3(4)-β-glucanases (E.C. 3.2.1.6).

In one embodiment (particularly for feed applications) the other component may be a mannanases (for example, a β-mannanase (E.C. 3.2.1.78)).

In one embodiment (particularly for feed applications) the other component may be a lipase (E.C. 3.1.1.3), a lipid acyltransferase (generally classified as E.C. 2.3.1.x), or a phospholipase (E.C. 3.1.1.4, E.C. 3.1.1.32 or E.C. 3.1.1.5); preferably a lipase (E.C. 3.1.1.3).

In one embodiment (particularly for feed applications) the other component may be a protease (for example, subtilisin (E.C. 3.4.21.62) or a bacillolysin (E.C. 3.4.24.28) or an alkaline serine protease (E.C. 3.4.21.x) or a keratinase (E.C. 3.4.x.x)).

In another embodiment the other component may be a further protease. Suitably, the further protease may be selected from the group consisting of: an aminopeptidase and a carboxypeptidase.

The term “aminopeptidase”, as used in this context, refers to an exopeptidase which is able to cleave single amino acids, di-amino acids or combinations thereof from the N-terminus of a protein and/or peptide substrate. Preferably, an aminopeptidase is able to cleave single amino acids only from the N-terminus of a protein and/or peptide substrate.

The aminopeptidase may be obtainable (preferably obtained) from Lactobacillus, suitably obtainable from Lactobacillus helveticus.

In one embodiment the aminopeptidase may be an aminopeptidase N (for example, PepN) (EC 3.4.11.2).

In one embodiment the aminopeptidase may comprise the sequence shown as SEQ ID NO: 5:

MAVKRFYKTFHPEHYDLRINVNRKNKTINGTSTITGDVIENPVFINQKFM
TIDSVKVDGKNVDFDVIEKDEAIKIKTGVTGKAVIEIAYSAPLTDTMMGI
YPSYYELEGKKKQIIGTQFETTFARQAFPCVDEPEAKATFSLALKWDEQD
GEVALANMPEVEVDKDGYHHFEETVRMSSYLVAFAFGELQSKTTHTKDGV
LIGVYATKAHKPKELDFALDIAKRAIEFYEEFYQTKYPLPQSLQLALPDF
SAGAMENWGLVTYREAYLLLDPDNTSLEMKKLVATVITHELAHQWFGDLV
TMKWWDNLWLNESFANMMEYLSVDGLEPDWHIWEMFQTSEAASALNRDAT
DGVQPIQMEINDPADIDSVFDGAIVYAKGSRMLVMVRSLLGDDALRKGLK
YYFDHHKFGNATGDDLWDALSTATDLDIGKIMHSWLKQPGYPVVNAFVAE
DGHLKLTQKQFFIGEGEDKGRQWQIPLNANFDAPKIMSDKEIDLGNYKVL
REEAGHPLRLNVGNNSHFIVEYDKTLLDDILSDVNELDPIDKLQLLQDLR
LLAEGKQISYASIVPLLVKFADSKSSLVINALYTTAAKLRQFVEPESNEE
KNLKKLYDLLSKDQVARLGWEVKPGESDEDVQIRPYELSASLYAENADSI
KAAHQIFTENEDNLEALNADIRPYVLINEVKNFGNAELVDKLIKEYQRTA
DPSYKVDLRSAVTSTKDLAAIKAIVGDFENADVVKPQDLCDWYRGLLANH
YGQQAAWDWIREDWDWLDKTVGGDMEFAKFITVTAGVFHTPERLKEFKEF
FEPKINVPLLSREIKMDVKVIESKVNLIEAEKDAVNDAVAKAID

The term “carboxypeptidase”, as used herein, has its usual meaning in the art and refers to an exopeptidase that is capable of cleaving n amino acids from the C-terminus of a peptide and/or protein substrate. In one embodiment n may be at least 1, suitably n may be at least 2. In other embodiments n may be at least 3, suitably at least 4.

In other embodiments, the tripeptidyl peptidase (optionally in combination with an endoprotease) may be used with one or more further exopeptidase.

In one embodiment the tripeptidyl peptidase (optionally in combination with an endoprotease) is not combined with (or used in combination with) a proline-specific exopeptidase.

In a particularly preferred embodiment, the tripeptidyl peptidase may not be combined with an enzyme having the following polypeptide sequence (SEQ ID NO: 6):

MRTAAASLTLAATCLFELASALMPRAPLIPAMKAKVALPSGNATFEQYI
DHNNPGLGTFPQRYWYNPEFWAGPGSPVLLFTPGESDAADYDGFLTNKT
IVGRFAEEIGGAVILLEHRYWGASSPYPELTTETLQYLTLEQSIADLVH
FAKTVNLPFDEIHSSNADNAPWVMTGGSYSGALAAWTASIAPGTFWAYH
ASSAPVQAIYDFWQYFVPVVEGMPKNCSKDLNRVVEYIDHVYESGDIER
QQEIKEMFGLGALKHFDDFAAAITNGPWLWQDMNFVSGYSRFYKFCDAV
ENVTPGAKSVPGPEGVGLEKALQGYASWFNSTYLPGSCAEYKYWTDKDA
VDCYDSYETNSPIYTDKAVNNTSNKQWTWFLCNEPLFYWQDGAPKDEST
IVSRIVSAEYWQRQCHAYFPEVNGYTFGSANGKTAEDVNKWTKGWDLTN
TTRLIWANGQFDPWRDASVSSKTRPGGPLQSTEQAPVHVIPGGFHCSDQ
WLVYGEANAGVQKVIDEEVAQIKAWVAEYPKYRKP

In one embodiment, the additional component may be a stabiliser or an emulsifier or a binder or carrier or an excipient or a diluent or a disintegrant.

The term “stabiliser”, as used herein, is defined as an ingredient or combination of ingredients that keeps a product (e.g. a feed product) from changing over time.

The term “emulsifier”, as used herein, refers to an ingredient (for example, a feed ingredient) that prevents the separation of emulsions. Emulsions are two immiscible substances, one present in droplet form, contained within the other. Emulsions can consist of oil-in-water, where the droplet or dispersed phase is oil and the continuous phase is water; or water-in-oil, where the water becomes the dispersed phase and the continuous phase is oil. Foams, which are gas-in-liquid, and suspensions, which are solid-in-liquid, can also be stabilised through the use of emulsifiers.

As used herein, the term “binder” refers to an ingredient (for example, a feed ingredient) that binds the product together through a physical or chemical reaction. For instance, during “gelation” water is absorbed and provides a binding effect. However, binders can absorb other liquids, such as oils, holding them within the product. In the context of the present compositions and methods, binders would typically be used in solid or low-moisture products for instance baking products: pastries, doughnuts, bread and others. Examples of granulation binders include one or more of: polyvinylpyrrolidone, hydroxypropylmethylcellulose (HPMC), hydroxypropylcellulose (HPC), sucrose, maltose, gelatin and acacia.

As used herein, “carriers” mean materials suitable for administration of the enzyme and include any such material known in the art such as, for example, any liquid, gel, solvent, liquid diluent, solubilizer, or the like, which is non-toxic and which does not interact with any components of the composition in a deleterious manner.

A method for preparing a composition is provided (e.g. a feed additive composition) comprising admixing a present feed additive (and preferably corn or a corn by-product) with at least one physiologically acceptable carrier selected from at least one of maltodextrin, limestone (calcium carbonate), cyclodextrin, wheat or a wheat component, sucrose, starch, Na2SO4, Talc, PVA, sorbitol, benzoate, sorbate, glycerol, sucrose, propylene glycol, 1,3-propane diol, glucose, parabens, sodium chloride, citrate, acetate, phosphate, calcium, metabisulfite, formate, and mixtures thereof.

Examples of “excipients” include one or more of: microcrystalline cellulose and other celluloses, lactose, sodium citrate, calcium carbonate, dibasic calcium phosphate, glycine, starch, and high molecular weight polyethylene glycols.

Examples of “disintegrants” include one or more of: starch (preferably corn, potato or tapioca starch), sodium starch glycollate, croscarmellose sodium, and certain complex silicates.

Examples of “diluents” include one or more of: water, ethanol, propylene glycol, glycerin, and combinations thereof.

The other components may be used simultaneously (for example, when they are in admixture together or even when they are delivered by different routes) or sequentially (for example, they may be delivered by different routes) to the present feed additive.

In one preferred embodiment, the feed additive composition, or feed ingredient, or feed or feedstuff or premix does not comprise chromium or organic chromium.

In one preferred embodiment, the feed additive composition, or feed ingredient, or feed or feedstuff or premix does not contain sorbic acid.

### Packaging

In one embodiment, the tripeptidyl peptidase and endoprotease and/or the composition and/or food and/or feed additive composition and/or hydrolysate and/or foodstuff and/or feedstuff are packaged.

In one preferred embodiment, the tripeptidyl peptidase and endoprotease and/or the composition and/or food and/or feed additive composition and/or hydrolysate and/or foodstuff and/or feedstuff is packaged in a bag, such as a paper bag.

In an alternative embodiment, the tripeptidyl peptidase and endoprotease and/or the composition and/or food and/or feed additive composition and/or hydrolysate and/or foodstuff and/or feedstuff may be sealed in a container. Any suitable container may be used.

### Foodstuff

The term “foodstuff” is used synonymously herein with “food”.

As used herein, the term “foodstuff” is used to refer to food for humans.

The food may be in the form of a solution or as a solid—depending on the use and/or the mode of application and/or the mode of administration.

When used as—or in the preparation of—a food—such as functional food—the hydrolysate and/or composition and/or food additive composition of the present invention may be used in conjunction with one or more of: a nutritionally acceptable carrier, a nutritionally acceptable diluent, a nutritionally acceptable excipient, a nutritionally acceptable adjuvant or a nutritionally active ingredient.

In one embodiment, a foodstuff is provided comprising a hydrolysate according to the invention. The foodstuff may additionally comprise a tripeptidyl peptidase (such as one obtainable by any of the methods herein), optionally in combination with an endoprotease.

Suitably the foodstuff may comprise at least one tripeptidyl peptidase comprising an amino acid sequence selected fromSEQ ID No. 3, SEQ ID No. 4, or a functional fragment thereof or an amino acid sequence having at least 70% identity therewith.

In another embodiment, a method is provided for the production of a foodstuff comprising contacting a food component with a hydrolysate of the invention or a composition and/or food additive composition of the invention.

Where a food component is contacted with a composition and/or food additive composition, suitably the food component may also be contacted with an endoprotease.

The present compositions can be used in the preparation of food products such as one or more of: jams, marmalades, jellies, dairy products (such as milk or cheese), meat products, poultry products, fish products and bakery products.

By way of example, the present compositions can be used as ingredients to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, breakfast cereals, instant noodles and cup noodles, instant soups and cup soups, balanced foods and drinks, sweeteners, texture improved snack bars, fibre bars, bake stable fruit fillings, care glaze, chocolate bakery filling, cheese cake flavoured filling, fruit flavoured cake filling, cake and doughnut icing, heat stable bakery filling, instant bakery filling creams, filing for cookies, ready-to-use bakery filling, reduced calorie filling, adult nutritional beverage, acidified soy/juice beverage, aseptic/retorted chocolate drink, bar mixes, beverage powders, calcium fortified soy and chocolate milk, and calcium fortified coffee beverages.

The present composition can further be used as an ingredient in food products such as American cheese sauce, anti-caking agent for grated & shredded cheese, chip dip, cream cheese, dry blended whip topping fat free sour cream, freeze/thaw dairy whipping cream, freeze/thaw stable whipped tipping, low fat & lite natural cheddar cheese, low fat Swiss style yoghurt, aerated frozen desserts, and novelty bars, hard pack ice cream, label friendly, improved economics & indulgence of hard pack ice cream, low fat ice cream: soft serve, barbecue sauce, cheese dip sauce, cottage cheese dressing, dry mix Alfredo sauce, mix cheese sauce, dry mix tomato sauce, and others.

For certain aspects, preferably the foodstuff is a beverage.

Preferably the foodstuff may be a bakery product—such as bread, Danish pastry, biscuits or cookies.

In another embodiment, a method of preparing a food or a food ingredient is provided, the method comprising admixing 5-KGA produced by the process of the present invention or the composition according to the present invention with another food ingredient. In another embodiment, a method for preparing or a food ingredient is also provided.

The foodstuff may be a dairy product, a whey-protein product, a bakery product, a fermentation product, a performance food, a baby food, a beverage, a shake or a casing.

Suitably the dairy product may be a milk-based product. Such milk-based products may comprise one or more milk proteins or fragments thereof.

Preferably the dairy (e.g. milk-based product) may be an infant formula.

Suitably the bakery product may be a bread product.

Suitably a fermentation product may be a soy-based fermentation product.

### Food Ingredient

The present hydrolysate and/or present food additive composition may be used as a food ingredient.

As used herein, the term “food ingredient” includes a formulation which is or can be added to functional foods or foodstuffs as a nutritional supplement and/or fiber supplement. The term food ingredient as used here also refers to formulations which can be used at low levels in a wide variety of products that require gelling, texturizing, stabilising, suspending, film-forming and structuring, retention of juiciness and improved mouthfeel, without adding viscosity.

The food ingredient may be in the form of a solution or as a solid—depending on the use and/or the mode of application and/or the mode of administration.

### Food Supplements

The hydrolysate and/or composition and/or food additive composition may be—or may be added to—food supplements.

### Functional Foods

The present composition(s) may be—or may be added to—functional foods.

As used herein, the term “functional food” means food which is capable of providing not only a nutritional effect and/or a taste satisfaction, but is also capable of delivering a further beneficial effect to consumer.

Accordingly, functional foods are ordinary foods that have components or ingredients (such as those described herein) incorporated into them that impart to the food a specific functional—for example, medical or physiological benefit—other than a purely nutritional effect.

Although there is no legal definition of a functional food, most of the parties with an interest in this area agree that they are foods marketed as having specific health effects.

Some functional foods are nutraceuticals. As used herein, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

Surveys have suggested that consumers place the most emphasis on functional food claims relating to heart disease. Preventing cancer is another aspect of nutrition which interests consumers a great deal, but interestingly this is the area that consumers feel they can exert least control over. In fact, according to the World Health Organization, at least 35% of cancer cases are diet-related. Furthermore, claims relating to osteoporosis, gut health and obesity effects are also key factors that are likely to incite functional food purchase and drive market development.

### Feed

The present feed additive composition may be used as—or in the preparation of—a feed.

In one embodiment, a feedstuff is provided comprising a hydrolysate as described herein. The feedstuff may additionally comprise a tripeptidyl peptidase (such as one obtainable by any of the methods herein), optionally in combination with an endoprotease.

Suitably, the feedstuff may comprise at least one tripeptidyl peptidase comprising an amino acid sequence selected from SEQ ID No. 3, SEQ ID No. 4 or any functional fragment thereof or an amino acid sequence having at least 70% identity therewith.

In another embodiment, a method is also provided for the production of a feedstuff comprising contacting a feed component with a hydrolysate as described herein.

As used herein, the term “feed” is used synonymously with “feedstuff”.

The feed may be in the form of a solution or as a solid—depending on the use and/or the mode of application and/or the mode of administration.

When used as—or in the preparation of—a feed—such as functional feed—the composition may be used in conjunction with one or more of: a nutritionally acceptable carrier, a nutritionally acceptable diluent, a nutritionally acceptable excipient, a nutritionally acceptable adjuvant, a nutritionally active ingredient.

In a preferred embodiment, the present feed additive composition is admixed with a feed component to form a feedstuff.

The term “feed component”, as used herein, means all or part of the feedstuff. Part of the feedstuff may mean one constituent of the feedstuff or more than one constituent of the feedstuff, e.g. 2, 3 or 4. In one embodiment, the term “feed component” encompasses a premix or premix constituents.

In one embodiment, a feed additive composition is provided comprising a tripeptidyl peptidase and one or more ingredients selected from the group consisting of: polyols, such as glycerol and/or sorbitol; sugars, such as glucose, fructose, sucrose, maltose, lactose and trehalose; salts, such as NaCl, KCl, CaCl2, Na2SO4 or other food grade salts; a preservative, e.g. sodium benzoate and/or potassium sorbate; or combinations thereof (optionally in combination with an endoprotease) may be admixed with at least one protein or portion thereof is an animal protein or a vegetable protein (e.g. selected from one or more of a gliadin, a beta-casein, a beta-lactoglobulin or an immunogenic fragment of a gliadin, a beta-casein, a beta-lactoglobulin, glycinin, beta-conglycinin, cruciferin, napin, collagen, whey protein, fish protein, meat protein, egg protein, soy protein a hordein or grain protein), preferably comprised in corn, soybean meal, corn dried distillers grains with solubles (DDGS), wheat, wheat proteins including gluten, wheat by products, wheat bran, corn by products including corn gluten meal, barley, oat, rye, triticale, full fat soy, animal by-product meals, an alcohol-soluble protein (preferably a zein (e.g. a maize zein maize) and/or a kafirin (e.g. from sorghum)), a protein from oil seeds (preferably from soybean seed proteins, sun flower seed proteins, rapeseed proteins, canola seed proteins or combinations thereof) or any combination thereof.

Preferably the feed may be a fodder, or a premix thereof, a compound feed, or a premix thereof. In one embodiment, the feed additive composition may be admixed with a compound feed, a compound feed component or to a premix of a compound feed or to a fodder, a fodder component, or a premix of a fodder.

The term fodder as used herein means any food which is provided to an animal (rather than the animal having to forage for it themselves). Fodder encompasses plants that have been cut.

The term fodder includes hay, straw, silage, compressed and pelleted feeds, oils and mixed rations, and also sprouted grains and legumes.

Fodder may be obtained from one or more of the plants selected from: alfalfa (Lucerne), barley, birdsfoot trefoil, brassicas, Chau moellier, kale, rapeseed (canola), rutabaga (swede), turnip, clover, alsike clover, red clover, subterranean clover, white clover, grass, false oat grass, fescue, Bermuda grass, brome, heath grass, meadow grasses (from naturally mixed grassland swards, orchard grass, rye grass, Timothy-grass, corn (maize), millet, oats, sorghum, soybeans, trees (pollard tree shoots for tree-hay), wheat, and legumes.

The term “compound feed” means a commercial feed in the form of a meal, a pellet, nuts, cake or a crumble. Compound feeds may be blended from various raw materials and additives. These blends are formulated according to the specific requirements of the target animal.

Compound feeds can be complete feeds that provide all the daily required nutrients, concentrates that provide a part of the ration (protein, energy) or supplements that only provide additional micronutrients, such as minerals and vitamins.

The main ingredients used in compound feed are the feed grains, which include corn, wheat, rye, maize, soybeans, sorghum, oats, and barley.

Suitably a premix as referred to herein may be a composition composed of microingredients such as vitamins, minerals, chemical preservatives, antibiotics, fermentation products, and other essential ingredients. Premixes are usually compositions suitable for blending into commercial rations.

Any feedstuff may comprise one or more feed materials selected from the group comprising a) cereals, such as small grains (e.g., wheat, barley, rye, oats and combinations thereof) and/or large grains such as maize or sorghum; b) by products from plants, such as Distillers Dried Grain Solubles (DDGS), wheat bran, wheat middlings, wheat shorts, rice bran, rice hulls, oat hulls, palm kernel, citrus pulp, corn fibre, corn germ meal, corn bran, Hominy feed, corn gluten feed, gluten meal, wheat shorts, wheat middlings or combinations thereof; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

A feedstuff may contain at least 30%, at least 40%, at least 50% or at least 60% by weight corn and soybean meal or corn and full fat soy, or wheat meal or sunflower meal.

In addition or in the alternative, a feedstuff may comprise at least one high fibre feed material and/or at least one by-product of the at least one high fibre feed material to provide a high fibre feedstuff. Examples of high fibre feed materials include: wheat, barley, rye, oats, by products from plants (e.g. cereals), such as Distillers Dried Grain Solubles (DDGS), wheat bran, wheat middlings, wheat shorts, rice bran, rice hulls, oat hulls, palm kernel, citrus pulp, corn fibre, corn germ meal, corn bran, Hominy feed, corn gluten feed, gluten meal, wheat shorts, wheat middlings or combinations thereof. Some protein sources may also be regarded as high fibre: protein obtained from sources such as sunflower, lupin, fava beans and cotton.

The feed may be one or more of the following: a compound feed and premix, including pellets, nuts or (cattle) cake; a crop or crop residue: corn, soybeans, sorghum, oats, barley, corn stover, copra, straw, chaff, sugar beet waste; fish meal; freshly cut grass and other forage plants; meat and bone meal; molasses; oil cake and press cake; oligosaccharides; conserved forage plants: hay and silage; seaweed; seeds and grains, either whole or prepared by crushing, milling etc.; sprouted grains and legumes; yeast extract.

The term “feed”, as used herein, also encompasses in some embodiments pet food. A pet food is plant or animal material intended for consumption by pets, such as dog food or cat food. Pet food, such as dog and cat food, may be either in a dry form, such as kibble for dogs, or wet canned form. Cat food may contain the amino acid taurine.

The term “feed” also encompasses in some embodiments fish food. A fish food normally contains macro nutrients, trace elements and vitamins necessary to keep captive fish in good health. Fish food may be in the form of a flake, pellet or tablet. Pelleted forms, some of which sink rapidly, are often used for larger fish or bottom feeding species. Some fish foods also contain additives, such as beta carotene or sex hormones, to artificially enhance the colour of ornamental fish.

The term “feed” also encompasses in some embodiment bird food. Bird food includes food that is used both in birdfeeders and to feed pet birds. Typically bird food comprises of a variety of seeds, but may also encompass suet (beef or mutton fat).

As used herein the term “contacting” refers to the indirect or direct application of the composition of the present invention to the product (e.g. the feed). Examples of the application methods which may be used, include, but are not limited to, treating the product in a material comprising the feed additive composition, direct application by mixing the feed additive composition with the product, spraying the feed additive composition onto the product surface or dipping the product into a preparation of the feed additive composition.

In one embodiment, the present feed additive composition is preferably admixed with the product (e.g. feedstuff). Alternatively, the feed additive composition may be included in the emulsion or raw ingredients of a feedstuff.

For some applications, it is important that the composition is made available on or to the surface of a product to be affected/treated. This allows the composition to impart one or more of the following favourable characteristics: biophysical characteristic is selected from the group consisting of one or more of the following: performance of the animal, growth performance of an animal, feed conversion ratio (FCR), ability to digest a raw material (e.g. nutrient digestibility, including starch , fat, protein, fibre digestibility), nitrogen digestibility (e.g. ileal nitrogen digestibility) and digestible energy (e.g. ileal digestible energy) nitrogen retention, carcass yield, growth rate, weight gain, body weight, mass, feed efficiency, body fat percentage, body fat distribution, growth, egg size, egg weight, egg mass, egg laying rate, lean gain, bone ash %, bone ash mg, back fat %, milk output, milk fat %, reproductive outputs such as litter size, litter survivability, hatchability % and environmental impact, e.g. manure output and/or nitrogen excretion.

The present feed additive compositions may be applied to intersperse, coat and/or impregnate a product (e.g. feedstuff or raw ingredients of a feedstuff) with a controlled amount of enzyme(s).

Preferably, the present feed additive composition will be thermally stable to heat treatment up to about 70° C.; up to about 85° C.; or up to about 95° C. The heat treatment may be performed for up to about 1 minute; up to about 5 minutes; up to about 10 minutes; up to about 30 minutes; up to about 60 minutes. The term thermally stable means that at least about 75% of the enzyme components that were present/active in the additive before heating to the specified temperature are still present/active after it cools to room temperature. Preferably, at least about 80% of the enzyme components that were present and active in the additive before heating to the specified temperature are still present and active after it cools to room temperature.

In a particularly preferred embodiment, the feed additive composition is homogenized to produce a powder.

In an alternative preferred embodiment, the feed additive composition is formulated to granules as described in WO2007/044968 (referred to as TPT granules) incorporated herein by reference.

In another preferred embodiment, when the feed additive composition is formulated into granules the granules comprise a hydrated barrier salt coated over the protein core. The advantage of such salt coating is improved thermo-tolerance, improved storage stability and protection against other feed additives otherwise having adverse effect on the enzyme.

Preferably, the salt used for the salt coating has a water activity greater than 0.25 or constant humidity greater than 60% at 20° C.

Preferably, the salt coating comprises a Na2SO4.

The method of preparing a feed additive composition may also comprise the further step of pelleting the powder. The powder may be mixed with other components known in the art. The powder, or mixture comprising the powder, may be forced through a die and the resulting strands are cut into suitable pellets of variable length.

Optionally, the pelleting step may include a steam treatment, or conditioning stage, prior to formation of the pellets. The mixture comprising the powder may be placed in a conditioner, e.g. a mixer with steam injection. The mixture is heated in the conditioner up to a specified temperature, such as from 60-100° C., typical temperatures would be 70° C., 80° C., 85° C., 90° C. or 95° C. The residence time can be variable from seconds to minutes and even hours. Such as 5 seconds, 10 seconds, 15 seconds, 30 seconds, 1 minutes 2 minutes., 5 minutes, 10 minutes, 15 minutes, 30 minutes and 1 hour.

It will be understood that the present feed additive composition is suitable for addition to any appropriate feed material.

As used herein, the term feed material refers to the basic feed material to be consumed by an animal. It will be further understood that this may comprise, for example, at least one or more unprocessed grains, and/or processed plant and/or animal material such as soybean meal or bone meal.

As used herein, the term “feedstuff” refers to a feed material to which one or more feed additive compositions have been added.

It will be understood by the skilled person that different animals require different feedstuffs, and even the same animal may require different feedstuffs, depending upon the purpose for which the animal is reared.

Preferably, the feedstuff may comprise feed materials comprising maize or corn, wheat, barley, triticale, rye, rice, tapioca, sorghum, and/or any of the by-products, as well as protein rich components like soybean mean, rape seed meal, canola meal, cotton seed meal, sunflower seed mean, animal-by-product meals and mixtures thereof. More preferably, the feedstuff may comprise animal fats and/or vegetable oils.

Optionally, the feedstuff may also contain additional minerals such as, for example, calcium and/or additional vitamins.

Preferably, the feedstuff is a corn soybean meal mix.

Feedstuff is typically produced in feed mills in which raw materials are first ground to a suitable particle size and then mixed with appropriate additives. The feedstuff may then be produced as a mash or pellets; the later typically involves a method by which the temperature is raised to a target level and then the feed is passed through a die to produce pellets of a particular size. The pellets are allowed to cool. Subsequently liquid additives such as fat and enzyme may be added. Production of feedstuff may also involve an additional step that includes extrusion or expansion prior to pelleting—in particular by suitable techniques that may include at least the use of steam.

The feedstuff may be a feedstuff for a monogastric animal, such as poultry (for example, broiler, layer, broiler breeders, turkey, duck, geese, and waterfowl), swine (all age categories), a pet (for example dogs, cats) or fish, preferably the feedstuff is for poultry.

By way of example only a feedstuff for chickens, e.g. broiler chickens may be comprises of one or more of the ingredients listed in the table below, for example in the percentages (%) given in the table below:

| Ingredients | Starter (%) | Finisher (%) |
|---|---|---|
| Maize | 46.2 | 46.7 |
| Wheat Middlings | 6.7 | 10.0 |
| Maize DDGS | 7.0 | 7.0 |
| Soyabean Meal 48% CP | 32.8 | 26.2 |
| An/Veg Fat blend | 3.0 | 5.8 |
| L-Lysine HCl | 0.3 | 0.3 |
| DL-methionine | 0.3 | 0.3 |
| L-threonine | 0.1 | 0.1 |
| Salt | 0.3 | 0.4 |
| Limestone | 1.1 | 1.1 |
| Dicalcium Phosphate | 1.2 | 1.2 |
| Poultry Vitamins and Micro-minerals | 0.3 | 0.3 |

By way of example only the diet specification for chickens, such as broiler chickens, may be as set out in the Table below:

| Diet specification | | |
|---|---|---|
| Crude Protein (%) | 23.00 | 20.40 |
| Metabolizable Energy Poultry | 2950 | 3100 |
| (kcal/kg) | | |
| Calcium (%) | 0.85 | 0.85 |
| Available Phosphorus (%) | 0.38 | 0.38 |
| Sodium (%) | 0.18 | 0.19 |
| Dig. Lysine (%) | 1.21 | 1.07 |
| Dig. Methionine (%) | 0.62 | 0.57 |
| Dig. Methionine + Cysteine (%) | 0.86 | 0.78 |
| Dig. Threonine (%) | 0.76 | 0.68 |

By way of example only a feedstuff laying hens may be comprises of one or more of the ingredients listed in the table below, for example in the %ages given in the table below:

| Ingredient | Laying phase (%) |
|---|---|
| Maize | 10.0 |
| Wheat | 53.6 |
| Maize DDGS | 5.0 |
| Soybean Meal 48% CP | 14.9 |
| Wheat Middlings | 3.0 |
| Soybean Oil | 1.8 |
| L-Lysine HCl | 0.2 |
| DL-methionine | 0.2 |
| L-threonine | 0.1 |
| Salt | 0.3 |
| Dicalcium Phosphate | 1.6 |
| Limestone | 8.9 |
| Poultry Vitamins and Micro-minerals | 0.6 |

By way of example only the diet specification for laying hens may be as set out in the Table below:

| Diet specification | |
|---|---|
| Crude Protein (%) | 16.10 |
| Metabolizable Energy Poultry | 2700 |
| (kcal/kg) | |
| Lysine (%) | 0.85 |
| Methionine (%) | 0.42 |
| Methionine + Cysteine (%) | 0.71 |
| Threonine (%) | 0.60 |
| Calcium (%) | 3.85 |
| Available Phosphorus (%) | 0.42 |
| Sodium (%) | 0.16 |

By way of example only a feedstuff for turkeys may be comprises of one or more of the ingredients listed in the table below, for example in the percentages (%) given in the table below:

| | Phase 1 | Phase 2 | Phase 3 | Phase 4 |
|---|---|---|---|---|
| Ingredient | (%) | (%) | (%) | (%) |
| Wheat | 33.6 | 42.3 | 52.4 | 61.6 |
| Maize DDGS | 7.0 | 7.0 | 7.0 | 7.0 |
| Soyabean Meal 48% CP | 44.6 | 36.6 | 27.2 | 19.2 |
| Rapeseed Meal | 4.0 | 4.0 | 4.0 | 4.0 |
| Soyabean Oil | 4.4 | 4.2 | 3.9 | 3.6 |
| L-Lysine HCl | 0.5 | 0.5 | 0.4 | 0.4 |
| DL-methionine | 0.4 | 0.4 | 0.3 | 0.2 |
| L-threonine | 0.2 | 0.2 | 0.1 | 0.1 |
| Salt | 0.3 | 0.3 | 0.3 | 0.3 |
| Limestone | 1.0 | 1.1 | 1.1 | 1.0 |
| Dicalcium Phosphate | 3.5 | 3.0 | 2.7 | 2.0 |
| Poultry Vitamins and | 0.4 | 0.4 | 0.4 | 0.4 |
| Micro-minerals | | | | |

By way of example only the diet specification for turkeys may be as set out in the Table below:

Diet specification

| | | | | |
|---|---|---|---|---|
| Crude Protein (%) | 29.35 | 26.37 | 22.93 | 20.00 |
| Metabolizable Energy Poultry | 2.850 | 2.900 | 2.950 | 3.001 |
| (kcal/kg) | | | | |
| Calcium (%) | 1.43 | 1.33 | 1.22 | 1.02 |
| Available Phosphorus (%) | 0.80 | 0.71 | 0.65 | 0.53 |
| Sodium (%) | 0.16 | 0.17 | 0.17 | 0.17 |
| Dig. Lysine (%) | 1.77 | 1.53 | 1.27 | 1.04 |
| Dig. Methionine (%) | 0.79 | 0.71 | 0.62 | 0.48 |
| Dig. Methionine + Cysteine (%) | 1.12 | 1.02 | 0.90 | 0.74 |
| Dig. Threonine (%) | 1.03 | 0.89 | 0.73 | 0.59 |

By way of example only a feedstuff for piglets may be comprises of one or more of the ingredients listed in the table below, for example in the percentages (%) given in the table below:

| Ingredient | Phase 1 (%) | Phase 2 (%) |
|---|---|---|
| Maize | 20.0 | 7.0 |
| Wheat | 25.9 | 46.6 |
| Rye | 4.0 | 10.0 |
| Wheat middlings | 4.0 | 4.0 |
| Maize DDGS | 6.0 | 8.0 |
| Soyabean Meal 48% CP | 25.7 | 19.9 |
| Dried Whey | 10.0 | 0.0 |
| Soyabean Oil | 1.0 | 0.7 |
| L-Lysine HCl | 0.4 | 0.5 |
| DL-methionine | 0.2 | 0.2 |
| L-threonine | 0.1 | 0.2 |
| L-tryptophan | 0.03 | 0.04 |
| Limestone | 0.6 | 0.7 |
| Dicalcium Phosphate | 1.6 | 1.6 |
| Swine Vitamins and Micro-minerals | 0.2 | 0.2 |
| Salt | 0.2 | 0.4 |

By way of example only the diet specification for piglets may be as set out in the Table below:

| Diet specification | | |
|---|---|---|
| Crude Protein (%) | 21.50 | 20.00 |
| Swine Digestible Energy | 3380 | 3320 |
| (kcal/kg) | | |
| Swine Net Energy (kcal/kg) | 2270 | 2230 |
| Calcium (%) | 0.80 | 0.75 |
| Digestible Phosphorus (%) | 0.40 | 0.35 |
| Sodium (%) | 0.20 | 0.20 |
| Dig. Lysine (%) | 1.23 | 1.14 |
| Dig. Methionine (%) | 0.49 | 0.44 |
| Dig. Methionine + Cysteine (%) | 0.74 | 0.68 |
| Dig. Threonine (%) | 0.80 | 0.74 |

By way of example only a feedstuff for grower/finisher pigs may be comprises of one or more of the ingredients listed in the table below, for example in the percentages (%) given in the table below:

| Ingredient | Grower/Finisher (%) |
|---|---|
| Maize | 27.5 |
| Soyabean Meal 48% CP | 15.4 |
| Maize DDGS | 20.0 |
| Wheat bran | 11.1 |
| Rice bran | 12.0 |
| Canola seed meal | 10.0 |
| Limestone | 1.6 |
| Dicalcium phosphate | 0.01 |
| Salt | 0.4 |
| Swine Vitamins and Micro-minerals | 0.3 |
| Lysine-HCl | 0.2 |
| Vegetable oil | 0.5 |

By way of example only the diet specification for grower/finisher pigs may be as set out in the Table below:

| Diet specification | |
|---|---|
| Crude Protein (%) | 22.60 |
| Swine Metabolizable Energy | 3030 |
| (kcal/kg) | |
| Calcium (%) | 0.75 |
| Available Phosphorus (%) | 0.29 |
| Digestible Lysine (%) | 1.01 |
| Dig. Methionine + Cysteine (%) | 0.73 |
| Digestible Threonine (%) | 0.66 |

### Meat Based Food/Feed Product

The hydrolysate may be used in the manufacture of a meat based food/feed product.

A “meat based food product” and “meat based feed product” is any product based on meat.

The meat based food product is suitable for human and/or animal consumption as a food and/or a feed. In one embodiment, the meat based food product is a feed product for feeding animals, such as for example a pet food product. In another embodiment, the meat based food product is a food product for humans.

A meat based food/feed product may comprise non-meat ingredients such as for example water, salt, flour, milk protein, vegetable protein, starch, hydrolysed protein, phosphate, acid, spices, colouring agents and/or texturizing agents.

A meat based food/feed product preferably comprises between 5-90% (weight/weight) meat. In some embodiments, the meat based food product may comprise at least 30% (weight/weight) meat, such as at least 50%, at least 60% or at least 70% meat.

In some embodiments, the meat based food/feed product is a cooked meat, such as ham, loin, picnic shoulder, bacon and/or pork belly for example.

The meat based food/feed product may be one or more of the following:

Dry or semi-dry cured meats—such as fermented products, dry-cured and fermented with starter cultures, for example dry sausages, salami, pepperoni and dry ham;

Emulsified meat products (e.g. for cold or hot consumption), such as mortadella, frankfurter, luncheon meat and pâté;

Fish and seafood, such as shrimps, salmon, reformulated fish products, frozen cold-packed fish;

Fresh meat muscle, such as whole injected meat muscle, for example loin, shoulder ham, marinated meat;

Ground and/or restructured fresh meat—or reformulated meat, such as upgraded cut-away meat by cold setting gel or binding, for example raw, uncooked loin chops, steaks, roasts, fresh sausages, beef burgers, meat balls, pelmeni;

Poultry products—such as chicken or turkey breasts or reformulated poultry, e.g. chicken nuggets and/or chicken sausages;

Retorted products—autoclaved meat products, for example picnic ham, luncheon meat, emulsified products.

In one embodiment, the meat based food/feed product is a processed meat product, such as for example a sausage, bologna, meat loaf, comminuted meat product, ground meat, bacon, polony, salami or pate.

A processed meat product may be for example an emulsified meat product, manufactured from a meat based emulsion, such as for example mortadella, bologna, pepperoni, liver sausage, chicken sausage, wiener, frankfurter, luncheon meat, meat pate.

The meat based emulsion may be cooked, sterilised or baked, e.g. in a baking form or after being filled into a casing of for example plastic, collagen, cellulose or a natural casing. A processed meat product may also be a restructured meat product, such a for example restructured ham. A meat product of the invention may undergo processing steps such as for example salting, e.g. dry salting; curing, e.g. brine curing; drying; smoking; fermentation; cooking; canning; retorting; slicing and/or shredding.

In another embodiment, the food/feed product may be an emulsified meat product.

### Meat

The term “meat” as used herein means any kind of tissue derived from any kind of animal.

The term meat as used herein may be tissue comprising muscle fibres derived from an animal. The meat may be an animal muscle, for example a whole animal muscle or pieces cut from an animal muscle.

In another embodiment the meat may comprise inner organs of an animal, such as heart, liver, kidney, spleen, thymus and brain for example.

The term meat encompasses meat which is ground, minced or cut into smaller pieces by any other appropriate method known in the art.

The meat may be derived from any kind of animal, such as from cow, pig, lamb, sheep, goat, chicken, turkey, ostrich, pheasant, deer, elk, reindeer, buffalo, bison, antelope, camel, kangaroo; any kind of fish e.g. sprat, cod, haddock, tuna, sea eel, salmon, herring, sardine, mackerel, horse mackerel, saury, round herring, Pollack, flatfish, anchovy, pilchard, blue whiting, pacific whiting, trout, catfish, bass, capelin, marlin, red snapper, Norway pout and/or hake; any kind of shellfish, e.g. clam, mussel, scallop, cockle, periwinkle, snail, oyster, shrimp, lobster, langoustine, crab, crayfish, cuttlefish, squid, and/or octopus.

In one embodiment the meat is beef, pork, chicken, lamb and/or turkey.

### Biophysical Characteristic

Feeding an animal hydrolysate obtainable (or obtained) the present method(s) may improve a biophysical characteristic of animal so fed.

Suitably, the method and/or use may further comprising administering to an animal at least one feed component, at least one mineral, at least one vitamin or any combination thereof.

The term “administering”, as used herein, may mean feeding the animal the hydrolysate produced in accordance with the present method(s) before, after or simultaneously with a feedstuff (e.g. the animal's usual diet). Alternatively, the term “administering” as used herein may mean feeding the animal with a feedstuff or premix comprising said hydrolysate.

Alternatively (or additionally) the method and/or use may further comprise administering to an animal at least one endoprotease.

As used herein, “biophysical characteristic” means any biophysical property of an animal which improves its health and/or performance and/or output.

By way of example, the biophysical characteristic may be one or more selected from the group consisting of one or more of the following: performance of the animal, growth performance of an animal, feed conversion ratio (FCR), ability to digest a raw material (e.g. nutrient digestibility, including starch , fat, protein, fibre digestibility), nitrogen digestibility (e.g. ileal nitrogen digestibility) and digestible energy (e.g. ileal digestible energy), nitrogen retention, carcass yield, growth rate, weight gain, body weight, mass, feed efficiency, body fat percentage, body fat distribution, growth, egg size, egg weight, egg mass, egg laying rate, lean gain, bone ash %, bone ash mg, back fat %, milk output, milk fat %, reproductive outputs such as litter size, litter survivability, hatchability % and environmental impact, e.g. manure output and/or nitrogen excretion.

Suitably, the biophysical characteristic may be one or more selected from the group consisting of: feed conversion ratio, nitrogen digestibility (e.g. ileal nitrogen digestibility) and digestible energy (e.g. ileal digestible energy).

In a preferred embodiment, the biophysical characteristic may be the ability to digest a protein.

In one embodiment, the biophysical characteristic of the animal means the performance of the animal.

Suitably, administering to an animal a feed additive composition and/or feed and/or feedstuff and/or feed ingredient and/or premix may not substantially increase the incidence of necrotic enteritis in the animal when compared to an animal not fed with the feed additive composition and/or feed and/or feedstuff and/or feed ingredient and/or premix.

The term “substantially increase the incidence of necrotic enteritis” as used herein means that the incidence is not increased by more than about 20%, suitably not increased by more than about 10%. Preferably it is meant that the incidence of necrotic enteritis is not increased by more than about 5%, more preferably more than about 1%.

### Performance

“performance of the animal” may be determined by the feed efficiency and/or weight gain of the animal and/or by the feed conversion ratio and/or by the digestibility of a nutrient in a feed (e.g. amino acid digestibility) and/or digestible energy or metabolizable energy in a feed and/or by nitrogen retention.

Preferably, “performance of the animal” is determined by feed efficiency and/or weight gain of the animal and/or by the feed conversion ratio.

By “improved performance of the animal” it is meant that there is increased feed efficiency, and/or increased weight gain and/or reduced feed conversion ratio and/or improved digestibility of nutrients or energy in a feed and/or by improved nitrogen retention in the subject resulting from the use of the present hydrolysate or present feed additive composition compared with feeding the animal a diet without said hydrolysate or feed additive composition.

Preferably, by “improved animal performance” it is meant that there is increased feed efficiency and/or increased weight gain and/or reduced feed conversion ratio.

As used herein, the term “feed efficiency” refers to the amount of weight gain in an animal that occurs when the animal is fed ad-libitum or a specified amount of food during a period of time.

By “increased feed efficiency” it is meant that the use of the present hydrolysate or present feed additive composition in feed results in an increased weight gain per unit of feed intake compared with an animal fed with a feed which does not comprise the present hydrolysate or present feed additive composition.

### Feed Conversion Ratio (FCR)

As used herein, the term “feed conversion ratio” refers to the amount of feed fed to an animal to increase the weight of the animal by a specified amount.

An improved feed conversion ratio means a lower feed conversion ratio.

By “lower feed conversion ratio” or “improved feed conversion ratio” it is meant that the use of the present feed additive composition or present hydrolysate in feed results in a lower amount of feed being required to be fed to an animal to increase the weight of the animal by a specified amount compared to the amount of feed required to increase the weight of the animal by the same amount when feed which does not comprise the present hydrolysate or present feed additive composition is used.

### Nutrient Digestibility

“Nutrient digestibility”, as used herein, means the fraction of a nutrient that disappears from the gastro-intestinal tract or a specified segment of the gastrointestinal tract, e.g. the small intestine. Nutrient digestibility may be measured as the difference between what is administered to the subject and what comes out in the faeces of the subject, or between what is administered to the subject and what remains in the digesta on a specified segment of the gastro intestinal tract, e.g. the ileum.

Nutrient digestibility may be measured by the difference between the intake of a nutrient and the excreted nutrient by means of the total collection of excreta during a period of time; or with the use of an inert marker that is not absorbed by the animal, and allows the researcher calculating the amount of nutrient that disappeared in the entire gastro-intestinal tract or a segment of the gastro-intestinal tract. Such an inert marker may be titanium dioxide, chromic oxide or acid insoluble ash. Digestibility may be expressed as a percentage of the nutrient in the feed, or as mass units of digestible nutrient per mass units of nutrient in the feed.

Nutrient digestibility, as used herein, encompasses starch digestibility, fat digestibility, protein digestibility, and amino acid digestibility.

Suitably, use of a tripeptidyl peptidase according to the present methods and/or uses (optionally in combination with at least one endoprotease) increases protein and/or amino acid digestibility in an animal fed with the feed additive composition and/or feed ingredient and/or feed and/or feedstuff and/or premix.

“Energy digestibility”, as used herein, means the gross energy of the feed consumed minus the gross energy of the faeces or the gross energy of the feed consumed minus the gross energy of the remaining digesta on a specified segment of the gastro-intestinal tract of the animal, e.g. the ileum. Metabolizable energy as used herein refers to apparent metabolizable energy and means the gross energy of the feed consumed minus the gross energy contained in the faeces, urine, and gaseous products of digestion. Energy digestibility and metabolizable energy may be measured as the difference between the intake of gross energy and the gross energy excreted in the faeces or the digesta present in specified segment of the gastro-intestinal tract using the same methods to measure the digestibility of nutrients, with appropriate corrections for nitrogen excretion to calculate metabolizable energy of feed.

### Nitrogen Retention

“Nitrogen retention”, as used herein, means as subject's ability to retain nitrogen from the diet as body mass. A negative nitrogen balance occurs when the excretion of nitrogen exceeds the daily intake and is often seen when the muscle is being lost. A positive nitrogen balance is often associated with muscle growth, particularly in growing animals.

Nitrogen retention may be measured as the difference between the intake of nitrogen and the excreted nitrogen by means of the total collection of excreta and urine during a period of time. It is understood that excreted nitrogen includes undigested protein from the feed, endogenous proteinaceous secretions, microbial protein, and urinary nitrogen.

### Carcass Yield and Meat Yield

The term “carcass yield”, as used herein, means the amount of carcass as a proportion of the live body weight, after a commercial or experimental process of slaughter. The term “carcass” means the body of an animal that has been slaughtered for food, with the head, entrails, part of the limbs, and feathers or skin removed. The term meat yield as used herein means the amount of edible meat as a proportion of the live body weight, or the amount of a specified meat cut as a proportion of the live body weight.

### Weight Gain

A method of increasing weight gain in a subject is also provided, e.g. poultry or swine, comprising feeding said subject a feedstuff comprising the present feed additive composition.

An “increased weight gain” refers to an animal having increased body weight on being fed feed comprising the present hydrolysate or present feed additive composition compared with an animal being fed a feed without said hydrolysate or feed additive composition.

### Nonfood Products

In another embodiment, a nonfood product is also provided comprising the present hydrolysate.

The present hydrolysate obtainable (e.g. obtained) may be used in the manufacture of a topically applied product, such as a lotion, cream, ointment, rub, cleanser, or the like. Accordingly, such products comprising the hydrolyzed protein compositions described herein are herein contemplated. Such products are useful for example for therapeutic purposes, for example, to provide relief from dry skin, itching, discomfort, and the like.

These products preferably comprise, in addition to the hydrolyzed protein component, a lipid, wax, oil, water in oil emulsion, oil-in-water emulsion, or the like as a base. Typically, they may further comprise one or more fragrance components, as well as other ingredients such as surfactants or emulsifiers.

Cosmetic products and other appearance aids or beauty aids comprising the milk or whey protein hydrolysates described herein are also provided.

In one embodiment, the cosmetic product may be applied to the face, cheeks, lips, or eyes of a person. In another embodiment the product may be used anywhere on the body to help improve the cosmetic appearance of the skin or, for example, to diminish the appearance of wrinkles moles, freckles, scars, blemishes, and the like.

### Advantages

The inventors have shown for the first time that a tripeptidyl peptidase is highly advantageous for use in the preparation of hydrolysates at higher temperatures.

Advantageously, a tripeptidyl peptidase as described herein is capable of acting on a wide range of peptide and/or protein substrates and due to having such a broad substrate-specificity is not readily inhibited from cleaving substrates enriched in certain amino acids (e.g. lysine and/or arginine and/or glycine). The use of such a tripeptidyl peptidase therefore may efficiently and/or rapidly breakdown protein substrates (e.g. present in a substrate for preparation of a hydrolysate).

In another embodiment, a thermostable tripeptidyl peptidases are provided which are less prone to being denatured and/or will therefore retain activity for a longer period of time when compared to a non-thermostable variant.

Advantageously, the tripeptidyl peptidase may have activity in a pH range of about pH 7 and can therefore be used with an alkaline endoprotease. This means that changing the pH of the reaction medium comprising the protein and/or peptide substrate for hydrolysate production is not necessary between enzyme treatments. In other words, it allows the tripeptidyl peptidase and the endoprotease to be added to a reaction simultaneously, which may make the process for producing the hydrolysate quicker and/or more efficient and/or more cost-effective. Moreover, this allows for a more efficient reaction as at lower pH values the substrate may precipitate out of solution and therefore not be cleaved.

A tripeptidyl peptidase having activity at an acidic pH can be used in combination with an acid endoprotease and advantageously does not require the pH of the reaction medium comprising the protein and/or peptide substrate for hydrolysate production to be changed between enzyme treatments. In other words, it allows the tripeptidyl peptidase and the endoprotease to be added to a reaction simultaneously, which may make the process for producing the hydrolysate quicker and/or more efficient and/or more cost-effective.

Advantageously, the tripeptidyl peptidase is capable of cleaving protein substrates associated with causing an immune response in sensitive individuals suffering from a disease, such as a milk protein allergy and/or a soy protein allergy.

Advantageously, the use of an endoprotease in combination with a tripeptidyl peptidase can increase the efficiency of substrate cleavage. Without wishing to be bound by theory, it is believed that an endoprotease is able to cleave a peptide and/or protein substrate at multiple regions away from the C or N-terminus, thereby producing more N-terminal ends for the tripeptidyl peptidase to use as a substrate, thereby advantageously increasing reaction efficiency and/or reducing reaction times.

Use of an endoprotease, a tripeptidyl peptidase and a further component e.g. carboxypeptidase and/or aminopeptidase has many advantages:
it allows for the efficient production of single amino acids and/or dipeptides and/or tripeptides which can efficiently be absorbed by a subject (e.g. due to having a better osmotic potential for uptake);
a protein and/or peptide substrate may be more efficiently and/or more quickly digested;
reduced end-point inhibition (i.e. inhibition by its reaction products) of a the tripeptidyl peptidase, particularly when used in vitro, such as in the manufacture of a hydrolysate by digesting the tripeptides into single amino acids and/or dipeptides; and/or
synergistic and/or additive activity on substrates containing high levels of lysine, arginine and/or glycine.

### Additional Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure which can be had by reference to the specification as a whole. Accordingly, the terms defined immediately below are more fully defined by reference to the specification as a whole.

Amino acids are referred to herein using the name of the amino acid, the three letter abbreviation or the single letter abbreviation.

In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to understand that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a tripeptidyl peptidase”, “an endoprotease” or “an enzyme” includes a plurality of such candidate agents and reference to “the feed”, “the feedstuff”, “the premix” or “the feed additive composition” includes reference to one or more feeds, feedstuffs, premixes and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### EXAMPLES

### Example 1

### Cloning and Expression of a Tripeptidyl Peptidase (TRI039) in Trichderma reesei.

A synthetic genes encoding tripeptidyl peptidase TRI039 was generated as a codon-optimized gene for expression in Trichderma reesei. The predicted secretion signal sequences (SignalP 4.0: Discriminating signal peptides from transmembrane regions. Thomas Nordahl Petersen, Soren Brunak, Gunnar von Heijne & Henrik Nielsen. Nature Methods, (2011) 8:785-786) were replaced with the secretion signal sequence from the Trichderma reesei acidic fungal protease (AFP) and an intron from a Trichderma reesei glucoamylase gene (TrGA1).

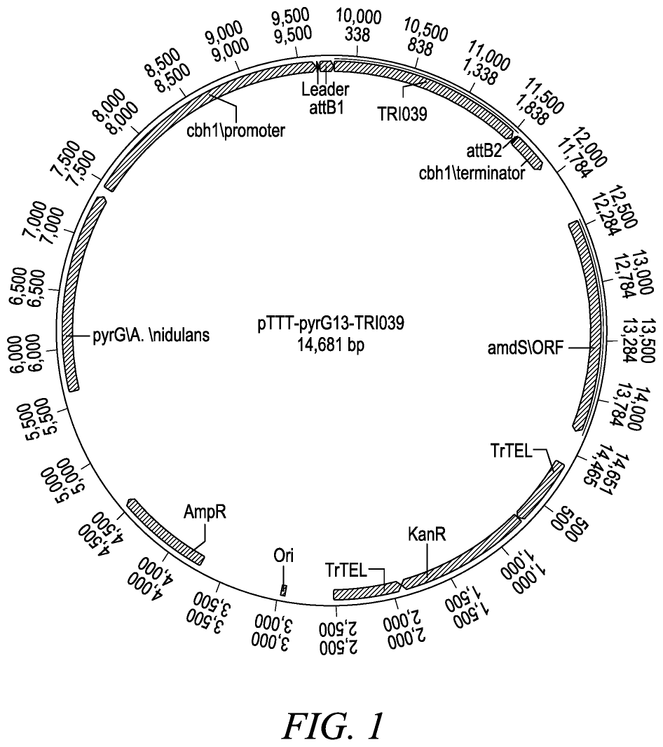

The synthetic gene was introduced into the destination vector pTTT-pyrG13 (as described in U.S. Pat. No. 8,592,194 B2, the teaching of which is incorporated herein by reference in its entirety) using LR Clonase™ enzyme mix (Thermo Fisher Scientific, Waltham, Mass.) resulting in the construction of expression vector pTTT-pyrG13 for the tripeptidyl peptidase. Expression vectors encoding SEQ ID No. 3 and SEQ ID No. 4 (TR1039) are shown in FIG. 1.

The expression vectors (5-10 μg) were transformed individually into a suitable Trichderma reesei strain using PEG mediated protoplast transformation essentially as described in U.S. Pat. No. 8,592,194 B2. Germinating spores were harvested by centrifugation, washed and treated with 45 mg/mL of lysing enzyme solution (Trichderma harzianum, Sigma-Aldrich, St. Louis, Mo.; L1412) to lyse the fungal cell walls. Further preparation of protoplasts was performed by a standard method, as described by Pennila et al. (Gene (1987) 61:155-164).

Spores were harvested using a solution of 0.85% NaCl, 0.015% TWEEN® 80. Spore suspensions were used to inoculate liquid cultures. Cultures were grown for 7 days at 28° C. and 80% humidity with shaking at 180 rpm. Culture supernatants were harvested by vacuum filtration and used to assay their performance as well as expression level.

### Example 2

### Purification and Characterization

### A. Purification of Tripeptidyl Peptidase

Desalting of samples was performed on PD10 column (GE Healthcare Life Sciences, Pittsburgh, Pa., USA) equilibrated with 20 mM Na-acetate, pH 4.5 (buffer A). For ion exchange chromatography on Source S15 HR25/5 (GE Healthcare Life Sciences) the column was equilibrated with buffer A. The desalted sample (7 mL) was applied to the column at a flow rate of 6 ml/min and the column was washed with buffer A. The bound proteins were eluted with a linier gradient of 0-0.35 M NaCl in 20 mM Na-acetate, pH 4.5 (35 min). During the entire run 10-mL fractions were collected. The collected samples were assay for tripeptidyl amino-activity as described below. Protein concentration was calculated based on the absorbance measure at 280 nm and the theoretical absorbance of the protein calculated using the ExPASy ProtParam tool.

### Example 3

### Whey Protein Hydrolysis (WPI) Employing Tripeptidyl Peptidase TRI039 at 40 and 50° C.

For WPI hydrolysis, LACPRODAN® 9224 (Arla Food Ingredients, Denmark) was employed, a 15% (w/w) WPI suspension was prepared in H₂O_{d} and adjusted to pH 6 using sodium hydroxide. To prevent microbial growth, 0.0285% (w/w) NaN₃ was added. Subsequently, 0.5% (w/w on protein substrate) FOODPRO® Alkaline Protease and 0.5% (w/w on protein substrate) FOODPRO® PNL was added and a volume of 200 μL of the WPI suspension was transferred into each of the 96 wells of a microtiter plate (MTP; VWR, Denmark). Following this, 5 μL of tripeptidyl peptidase TR1039 containing either 0, 2188 or 4376 nkat/mL were added to the particular wells of the MTP. Then, the MTP was sealed and placed in an incubator at 40 or 50° C. (iEMS incubator/shaker HT, Thermo Scientific, Denmark).

After 24 h of incubation and shaking at 400 rpm, the hydrolysis was stopped by addition of 20 μL of 2 M trichloroacetic acid (TCA; Sigma-Aldrich, Denmark), except for the reference (0 h) to which the TCA was added prior to endo- and exopeptidase addition. Unhydrolyzed, precipitated WPI was removed by filtration (0.22 μm; Corning 3504 filter plate, Corning Incorporated, USA). The filtered WPI hydrolysate was employed for o-phthaldehyde (OPA) derivatization (Nielsen, P. M., et al. (2001) Journal of Food Science 66(5): 642-646). The OPA derivatization was conducted according to Nielsen et al. (2001) with minor modifications. A sample volume of 25 μL was transferred to a well and 175 μL of OPA-reagent, dissolved in trisodium phosphate-dodecahydrate, was added subsequently. The measured absorptions at 340 nm in a MTP reader (VersaMax, Molecular Devices, Denmark) were transformed into serine equivalents employing a serine calibration curve (0-2 mM).

As shown in Table 1 the tripeptidyl amino-peptidase TR1039 gave 3.6-3.8 times higher hydrolysis at 50° C. compared to 40° C.

TABLE 1
Analysis of increase in DH (in %) of WPI
hydrolysate due to addition of TRI039

| | |
|---|---|
| Quantities of TRI039 | TRI039 (nkat) |

| activity used in the assey | 10.9 | 21.9 |
|---|---|---|
| 40° C. | 1.2 | 1.5 |
| 50° C. | 4.6 | 5.4 |

### REFERENCES

Nakadai, T., et al. (1973). “Purification and properties of leucine amino-peptidase I from Aspergillus oryzae.” Agricultural and Biological Chemistry 37(4): 757-765.
Nielsen, P. M., et al. (2001). “Improved method for determining food protein degree of hydrolysis.” Journal of Food Science 66(5): 642-646.
Stressler, T., et al. (2013). “Characterization of the Recombinant Exopeptidases PepX and PepN from Lactobacillus helveticus ATCC 12046 Important for Food Protein Hydrolysis.” PLoS ONE 8(7).
Wang, F., et al. (2012). “Biochemical and conformational characterization of a leucine amino-peptidase from Geobacillus thermodenitrificans NG80-2.” World Journal of Microbiology and Biotechnology 28(11): 3227-3237.

1. A method for the production of a hydrolysate comprising:
a) admixing at least one protein or a portion thereof with a tripeptidyl peptidase which:
i) comprises the amino acid sequence SEQ ID No. 3, SEQ ID No. 4 or a functional fragment thereof;
ii) comprises an amino acid having at least 70% identity to SEQ ID No. 3 or SEQ ID No. 4;
iii) is encoded by a nucleotide sequence comprising the sequence SEQ ID No. 1 or SEQ ID No. 2;
iv) is encoded by a nucleotide sequence which has at least about 70% identity to SEQ ID No. 1 or SEQ ID No. 2;
v) is encoded by a nucleotide sequence which hybridises to SEQ ID No. 1 or SEQ ID No. 2 under medium stringency conditions; or
vi) is encoded by a nucleotide sequence which differs from SEQ ID No. 1 or SEQ ID No. 2 due to degeneracy of the genetic code;
b) incubating at a temperature between 45° C. and 70° C., and
c) recovering the hydrolysate.

2. A method according to claim 1 wherein the temperature of the incubation is between 50° C. and 65° C.

3. A method according to claim 2 wherein the temperature of the incubation is between 55° C. and 65° C.

4. A method according to claim 3, wherein the method further comprises admixing the recovered hydrolysate with at least one feed or food ingredient.

5. A method according to claim 4 wherein the protein or portion thereof is further treated with an endoprotease.

6. A method according to claim 5 wherein the endoprotease and the tripeptidyl peptidase are added simultaneously.

7. A method according to claim 6 wherein the endoprotease and the tripeptidyl peptidase are added sequentially, e.g. with the tripeptidyl peptidase after the endoprotease.

8. A method according to claim 7 wherein the endoprotease and tripeptidyl peptidase are active at a similar pH range.

9. A method according to claim 8, wherein the endoprotease is an acid endoprotease.

10. A method according to claim 8, wherein the endoprotease is an alkaline endoprotease, preferably selected from a trypsin, a chymotrypsin, and a combination thereof.

11. A method according to claim 10 wherein the hydrolysate has a reduced immunogenicity in a subject predisposed to having an immune response to the at least one protein or portion thereof.

12. A method according to claim 11 wherein the at least one protein is an animal protein or a plant protein, preferably wherein the protein is one or more of a gliadin, a beta-casein, a beta-lactoglobulin or an immunogenic fragment of a gliadin, a beta-casein, a beta-lactoglobulin, whey protein, fish protein, meat protein, egg protein, soy protein, a hordein or grain protein.

13. A reaction system comprising at least one protein or a portion thereof and a tripeptidyl peptidase which:
a) comprises the amino acid sequence SEQ ID No. 3, SEQ ID No. 4 or a functional fragment thereof;
b) comprises an amino acid having at least 70% identity to SEQ ID No. 3 or SEQ ID No. 4;
c) is encoded by a nucleotide sequence comprising the sequence SEQ ID No. 1 or SEQ ID No. 2;
d) is encoded by a nucleotide sequence which has at least about 70% identity to SEQ ID No. 1 or SEQ ID No. 2;
e) is encoded by a nucleotide sequence which hybridises to SEQ ID No. 1 or SEQ ID No. 2 under medium stringency conditions; or
f) is encoded by a nucleotide sequence which differs from SEQ ID No. 1 or SEQ ID No. 2 due to degeneracy of the genetic code;
wherein the reaction system is maintained at a temperature between 45° C. and 70° C. for a sufficient period of time to allow production of a hydrolysate.

14. A reaction system according to claim 13 wherein the temperature is maintained between 50° C. and 65° C.

15. A reaction system according to claim 14 wherein the temperature is maintained between 55° C. and 65° C.

16. A reaction system according to claim 15 which further comprises an endoprotease.

17. A reaction system according to claim 16 wherein the endoprotease and tripeptidyl peptidase are active at a similar pH range.

18. A reaction system according to claim 17, wherein the endoprotease is an acid endoprotease.

19. A reaction system according to claim 18, wherein the endoprotease is an alkaline endoprotease, preferably selected from a trypsin, a chymotrypsin, and a combination thereof.

20. A reaction system according to claim 19 wherein the at least one protein is an animal protein or a plant protein, preferably wherein the protein is one or more of a gliadin, a beta-casein, a beta-lactoglobulin or an immunogenic fragment of a gliadin, a beta-casein, a beta-lactoglobulin, whey protein, fish protein, meat protein, egg protein, soy protein, a hordein or grain protein.

21. A method for the expression of a tripeptidyl peptidase, wherein said method comprises:
a) transforming a Trichderma host cell with a nucleic acid or vector comprising
i) the nucleotide sequence SEQ ID No. 1 or SEQ ID No. 2;
ii) a nucleotide sequence which has at least about 70% identity to SEQ ID No. 1 or SEQ ID No. 2;
iii) a nucleotide sequence which hybridises to SEQ ID No. 1 or SEQ ID No. 2 under medium stringency conditions; or
iv) a nucleotide sequence which differs from SEQ ID No. 1 or SEQ ID No. 2 due to degeneracy of the genetic code;
b) expressing the nucleic acid sequence or vector of step a); and
c) obtaining the tripeptidyl peptidase or a fermentate comprising said tripeptidyl peptidase and optionally isolating and/or purifying and/or packaging.

22. The method of claim 21, wherein the host cell is a Trichderma reesei host cell.

23. Use of a tripeptidyl peptidase which:
a) comprises the amino acid sequence SEQ ID No. 3, SEQ ID No. 4 or a functional fragment thereof;
b) comprises an amino acid having at least 70% identity to SEQ ID No. 3 or SEQ ID No. 4;
c) is encoded by a nucleotide sequence comprising the sequence SEQ ID No. 1 or SEQ ID No. 2;
d) is encoded by a nucleotide sequence which has at least about 70% identity to SEQ ID No. 1 or SEQ ID No. 2;
e) is encoded by a nucleotide sequence which hybridises to SEQ ID No. 1 or SEQ ID No. 2 under medium stringency conditions; or
f) is encoded by a nucleotide sequence which differs from SEQ ID No. 1 or SEQ ID No. 2 due to degeneracy of the genetic code;
in the manufacture of a hydrolysate at a temperature between 45° C. and 70° C.

24. The use according to claim 23 for reducing the immunogenicity of the hydrolysate in a subject predisposed to having an immune reaction to the untreated protein or portion thereof or for reducing bitterness of the hydrolysate.

25. (canceled)

26. (canceled)

27. A hydrolysate obtainable from the method of claim 1.

28. A feed additive composition or food additive composition comprising the hydrolysate of claim 27.

29. A method for producing a feedstuff or foodstuff comprising contacting a feed component or food component with the hydrolysate of claim 27.

30. A method according to claim 29 wherein the feedstuff or foodstuff is a dairy product, (preferably a milk-based product), a whey-protein product, a bakery product (preferably a bread product), a fermentation product (preferably a soy-based fermentation product), a sports nutrition product, a performance food, a beverage, a baby food, a food for elderly, a food for people in medical care, a shake, or a casing (preferably, a casing for beer or dairy).

31. A feedstuff or foodstuff comprising a hydrolysate according to claim 27.

32. A nonfood product comprising the hydrolysate according to claim 27, wherein the nonfood product is a cosmetic, a lotion, or a cleanser for use on human skin.

33. (canceled)
